SEQUENCE LISTING (84 sequences)

SEQ ID NO: 1 - DNA, 6394 bp, Homo sapiens
  CDS (124)..(5097)
ggacccactgggttgccaagctcgcgccggatgcggagcgcggtgctgccggtggagcttcaggtcttgatagactttctgtaaagaaggaatgatttggtgatggagtgttcccactgaccgatggactcaaagaagagaagctcaacagaggcagaaggatccaaggaaagaggcctggtccatatctggcaggcaggatcctttcccataacaccagagagattgccaggctggggaggaaagactgttttgcaggcagccctcggagtgaaacatggagttcttctgactgaagatggtgaggtctacagctttgggactcttccctggagaagtggaccagtggagatttgtccaagtagccccattctagaaaatgccctggttgggcaatatgttattactgtggcaacaggaagcttccatagtggagcagtgacagacaatggtgtcgcgtacatgtggggagagaattctgctggccagtgtgcagtagccaaccagcagtatgtgccggaaccaaatcctgtcagcattgctgattctgaggccagccctttgttagcagtcaggattttacagttggcgtgtggcgaggagcacactctggcattgtcaataagcagagagatttgggcatggggtaccggttgtcagttgggtctcattaccactgccttcccagtgacaaagccgcaaaaggtagaacatcttgctgggcgagtggtgcttcaagttgcctgtggtgctttccacagcttagcccttgtacaatgcctcccttcccaggatctgaagccagtcccagaacgatgcaaccagtgcagccagctcttgattactatgactgacaaagaagaccatgtgattatatcagacagtcattgttgcccattaggtgtgacactgacagaatctcaggcagaaaaccatgccagcactgctctcagcccctccactgaaacccttgacaggcaggaagaagtatttgagaacactcttgtagcaaatgatcagtctgttgctactgaactgaatgcagtaagtgctcagatcacaagcagcgatgccatgtcctctcaacaaaatgtcatgggaacaactgaaatttcctctgccagaaacataccatcataccctgacacccaagcagtcaatgaatacctacggaaactgtcagatcattcagtaagagaggactcagagcatggtgaaaagccaatgccatctcagcctcttttagaagaagcaattcctaatctccacagcccgcctaccacaagcacctcagccctaaacagcctggtggtctcttgtgcatctgctgttggtgtgagagtggctgctacttatgaagctggtgccttgtcactgaagaaagttatgaacttttatagtacaaccccttgtgaaactggagctcaggcaggcagtagtgccattggccccgaaggtttgaaagatagcagggaagaacaggttaaacaggaatcaatgcaaggaaagaaaagttcaagtcttgtggatatcagagaagaagaaacagagggaggcagtcgaagactctccctccctggattgttgtcacaagtttcccccaggctcttaagaaaggctgcacgggtgaaaacgaggacagtggttctgacccccacatacagtggagaagcagatgcgctcctgccttctctgagaacagaagtgtggacctgggggaaagggaaggaagggcagctggggcacggcgatgttctgcctaggcttcaaccgttgtgtgtaaaatgtctggatggcaaagaagtaatccatctggaggcaggtggttaccattctcttgcacttactgcgaaatcccaggtttactcatggggtagcaatacctttggtcaacttgggcattccgattttccaacaacagttcctcgtcttgcaaagataagcagtgaaaatggagtctggagcatagctgcaggcagggattattccctgtttttagtggatacagaagacttccagcctgggttatattacagtggccgacaggaccctacagaaggtgacaaccttccagagaatcacagtggttctaagactccagtacttctctcctgtagtaagcttggatatataagcagagtgacagcaggaaaagatagctatttagccttggtggataaaaacattatggggtatattgccagtctccacgagttagctactacagaaagacgattctattcaaaactaagtgatatcaaatctcagattctcaggcctcttctcagtttagaaaatttgggcactacaactacagtccagctgttgcaggaggtggctagccgattcagcaagctgtgttacctcattggtcagcatggagcctcattgagcagcttccttcatggggtaaaggaagccaggagtttggtcatcctgaagcattcaagtctcttcttggatagttatacagagtattgcacatctattacaaatttcctggttatgggaggattccagcttcttgctaagcctgccattgatttcctaaataaaaaccaagagctgttgcaagatttgtcagaagtgaatgacgaaaacactcagttgatggaaatactgaatactttgtttttcttgccaatcagacgacttcataattacgcaaaagttttgctaaagcttgctacttgttttgaagtggcatctccagaatatcagaaactgcaggattccagttcttgttatgagtgtcttgctctccatctcggcaggaaaaggaaggaagcagaatacacactgggcttctggaagaccttccccggaaaaatgacggattccttgaggaagccagagcgtcgactgctgtgtgagagtagtaaccgagccctgtctctgcagcatgctgggaggttttccgtgaattggttcattctctttaatgatgccctggtccatgcccagttctccacgcaccatgttttccctctggccacgctgtgggcagagccactgtctgaagaagctggtggtgtgaatggcttaaagataactacacctgaggagcagttcactctcatttcatctacaccccaggaaaagacaaagtggctacgagctataagccaagccgtagatcaggctttgagagggatgtctgatctccccccttatggaagtggtagcagtgttcagagacaggaaccacccatttcacgcagtgccaaatatactttctacaaggatcctcgcctaaaggatgccacctatgatggacgctggctttcagggaagcctcatggcagaggggttttgaagtggcctgatggaaagatgtattctggcatgttcaggaatggcttggaagatgggtatggagaatacagaatcccaaacaaggcaatgaacaaagaagaccattatgtgggccattggaaagaaggaaaaatgtgcggtcaaggagtctacagctatgcttctggtgaagtatttgagggctgttttcaagataatatgcgtcatggtcatggtcttctacgaagtgggaaattgacgtcctcttctcctagtatgttcattggccagtgggtaatggataagaaagcaggatatggtgtctttgatgatatcactaggggggaaaagtatatgggaatgtggcaagatgatgtgtgtcaagggaatggtgtggtggttacccagtttggattatactacgagggcaactttcaccttaataaaatgatgggaaatggggttttgctttccgaagatgatactatctatgaaggagaattttcagatgactggactcttagtggaaagggaacactgactatgccaaatggagactacattgaaggttattttagtggagaatggggatctgggataaaaatcactggaacctacttcaaacctagtctatatgagagtgataaagacagacctaaagttttcaggaagctaggaaacctggcagtgccagctgatgagaagtggaaagcggtgtttgacgaatgttggcgccaactgggctgtgagggcccaggccaaggggaagtttggaaagcatgggacaatattgctgtggccttgaccaccagtcggcgccagcacagagacagtccagaaatattgagtcgttcacagactcagacactagagagtttggaattcattccacagcatgttggtgccttctctgtggagaaatatgatgacatcaggaaatatttaataaaggcctgtgacactcctctgcaccccctgggcaggcttgtggagacactggttgcagtgtatagaatgacatacgtgggcgtaggagccaaccgcaggttattgcaggaggctgtaaaggagattaagtcctatcttaagcgaattttccagctggtgaggttcttatttcctgagctgcctgaagaaggcagcacaattcctctctctgctcctctgccaaccgaaaggaagtctttttgcactgggaagtcagattcccgatctgaatcaccagagccaggttatgtagtaacgagttctggattattgcttcctgtgctgctacctcggctctacccaccgctgtttatgctttatgctttggataatgatcgcgaggaagacatttactgggaatgtgtccttcgactaaataagcagccagatattgctctcctgggctttcttggggtgcagaggaaattttggccagcaaccttgtcaatccttggagagagtaaaaaggttttgccaaccacgaaagatgcttgttttgcctcagcagtagaatgtctgcagcagatcagcacaacatttaccccatcagacaaacttaaggtcatccagcagacttttgaggagatctctcagagtgtcctggcgtcactccacgaagacttcttgtggtccatggatgacttgtttcctgttttcttatatgtggtgctacgggccaggattaggaatttaggctctgaggtacacctcattgaggatctaatggacccctatcttcagcatggggaacagggtataatgttcaccaccttgaaggcatgttactaccagattcagcgtgagaagcttaactaggctgcataacagcttgaaaactggattatctactacagagtgttataacaccatctggagtcttcctgtagtggcaaaaaagaacagtgttgaaattggaaaggactttgtgttatttaggttgttagaatgagccttaccaataataagagccctgagcccagaaaaaaggactgtatagtttaaagggaggattgaaagggaggtaaaaaatcagattagaccagttcttggcctatgataagttccaaaaataccatttatctactatttgaaaaaagaagaggatatcccttcctacagtaaagggtatgtcagctacatgaagttgtaagaaaagcttccagtagagcttcttatattaaagaagttgatggatatttttgaatttctggtttgcctgaatccacctgcagttaccccgatccgtttgcaagaaccagatcgtacttgaaactatagtggccacactctgccttcctgagtcccttccagtcatgtgtgcatcatgtctctttgccaagggaggggagaaaggaacttttaaactgcagttttaactttttctaagctgtttcttgatgggagaggttctgtgcaaaactaccacattctgtccccaaaatgtggaatgcatccaaataggagtcttctgcctcttaacttaaaagaacataggaattttgtttttggtttctttatcatgctacagagagtgaatacactggaattcagacaccgactctgagctgctaggaacctcatttgtccatgtgcaaacgctgtattccaaggcctgtgaatggcagcctgaggaagttttgcatgcaggctgtgttttcgagcaggactaacaactgggaaataagcaaaaaactgcatcgatccccagcctggtgttgttcttccctatacttcacactgaactcaggatgggaagaaaaaggaaacaagctttggctttttccatctcaaaagtattgtggcacctcaacatttcagtgttttgctttttaaaaaatgccctattgtaagttgttggtttatactgtataagtaacactagtagctgttttgaataacataggtgctcttcctcatctcatctcctacaccgtggtgagcatacagagtgtcctgatttgtgttaagtgactgagaagatgttaattacttttgaaaaaggatcatggtttttgctctactttataatcaagacaagtgtttattaaaatactgttttggaatgttggctgtaatgtaacagcaattttcataataaaaggcattcatcttt

SEQ ID NO: 2 - protein, 1657 aa, Homo sapiens
MDSKKRSSTEAEGSKERGLVHIWQAGSFPITPERLPGWGGKTVLQAALGVKHGVLLTEDGEVYSFGTLPWRSGPVEICPSSPILENALVGQYVITVATGSFHSGAVTDNGVAYMWGENSAGQCAVANQQYVPEPNPVSIADSEASPLLAVRILQLACGEEHTLALSISREIWAWGTGCQLGLITTAFPVTKPQKVEHLAGRVVLQVACGAFHSLALVQCLPSQDLKPVPERCNQCSQLLITMTDKEDHVIISDSHCCPLGVTLTESQAENHASTALSPSTETLDRQEEVFENTLVANDQSVATELNAVSAQITSSDAMSSQQNVMGTTEISSARNIPSYPDTQAVNEYLRKLSDHSVREDSEHGEKPMPSQPLLEEAIPNLHSPPTTSTSALNSLVVSCASAVGVRVAATYEAGALSLKKVMNFYSTTPCETGAQAGSSAIGPEGLKDSREEQVKQESMQGKKSSSLVDIREEETEGGSRRLSLPGLLSQVSPRLLRKAARVKTRTVVLTPTYSGEADALLPSLRTEVWTWGKGKEGQLGHGDVLPRLQPLCVKCLDGKEVIHLEAGGYHSLALTAKSQVYSWGSNTFGQLGHSDFPTTVPRLAKISSENGVWSIAAGRDYSLFLVDTEDFQPGLYYSGRQDPTEGDNLPENHSGSKTPVLLSCSKLGYISRVTAGKDSYLALVDKNIMGYIASLHELATTERRFYSKLSDIKSQILRPLLSLENLGTTTTVQLLQEVASRFSKLCYLIGQHGASLSSFLHGVKEARSLVILKHSSLFLDSYTEYCTSITNFLVMGGFQLLAKPAIDFLNKNQELLQDLSEVNDENTQLMEILNTLFFLPIRRLHNYAKVLLKLATCFEVASPEYQKLQDSSSCYECLALHLGRKRKEAEYTLGFWKTFPGKMTDSLRKPERRLLCESSNRALSLQHAGRFSVNWFILFNDALVHAQFSTHHVFPLATLWAEPLSEEAGGVNGLKITTPEEQFTLISSTPQEKTKWLRAISQAVDQALRGMSDLPPYGSGSSVQRQEPPISRSAKYTFYKDPRLKDATYDGRWLSGKPHGRGVLKWPDGKMYSGMFRNGLEDGYGEYRIPNKAMNKEDHYVGHWKEGKMCGQGVYSYASGEVFEGCFQDNMRHGHGLLRSGKLTSSSPSMFIGQWVMDKKAGYGVFDDITRGEKYMGMWQDDVCQGNGVVVTQFGLYYEGNFHLNKMMGNGVLLSEDDTIYEGEFSDDWTLSGKGTLTMPNGDYIEGYFSGEWGSGIKITGTYFKPSLYESDKDRPKVFRKLGNLAVPADEKWKAVFDECWRQLGCEGPGQGEVWKAWDNIAVALTTSRRQHRDSPEILSRSQTQTLESLEFIPQHVGAFSVEKYDDIRKYLIKACDTPLHPLGRLVETLVAVYRMTYVGVGANRRLLQEAVKEIKSYLKRIFQLVRFLFPELPEEGSTIPLSAPLPTERKSFCTGKSDSRSESPEPGYVVTSSGLLLPVLLPRLYPPLFMLYALDNDREEDIYWECVLRLNKQPDIALLGFLGVQRKFWPATLSILGESKKVLPTTKDACFASAVECLQQISTTFTPSDKLKVIQQTFEEISQSVLASLHEDFLWSMDDLFPVFLYVVLRARIRNLGSEVHLIEDLMDPYLQHGEQGIMFTTLKACYYQIQREKLN

SEQ ID NO: 3 - protein, 49 aa, Homo sapiens
MDSKKRSSTEAEGSKERGLVHIWQAGSFPITPERLPGWGGKTVLQAPSE

SEQ ID NO: 4 - DNA, 6349 bp, Mus musculus
  CDS (124)..(5079)
ccacgcgtccggcggtgcagtcgggctcgcgccgggagaagagcgcggagctgcgggagcgtcaggtcttgagagagcttttgctaatgggatggtttggtgatggagtactcctcctgaccgatggactcaaagaagaaaagctcaacagaggcagaaggatccaaagaaagaggcctagtccatgtctggcaggcaggatccttttctctaacaccagagaggttgccaggctggggtggaaagacagttcttcaggcagcccttggtgtgaggcatggagttcttctgactgaagatggtgaggtctacagctttgggactcttccctggaaaagtgaatcagcagaaatttgtccaagcagcccccttctagaaagtgccctggttgggcatcatgttattactgtggcaacagggagcttccacagtggagcagtgacagagagcggggtggtgtacatgtggggagagaatgctgccgggcagtgtgcggtagctaaccagcagtatgtgccggagccgagtcctgtcagcatttctgactcggagaccagcccgtcattagcagttaggattctgcaattggcatgtggcgaggaacacacactggcattgtcactcagcagagagatctgggcatggggcaccggctgtcagctgggcctcatcaccaccactttcccagtgacaaagccacagaaggtggaacaccttgctggacgagtggtgctccaggtggcctgcggtgcattccacagccttgcacttgtgcagtgcctccctcctcaggatctgaagccagtcccagagagatgcaatcagtgcagccagctgctcatcaccatgacagacaaagaggaccatgtgataatatcggacagccattgctgccctttaggtgtgacattgtccgagtctcaagcagaaaagcatgccagccctgctcccagccctcacccagaggcactggatgagcagggagaggtgtttgagaacacggtggtagaagctgaactgaacatgggaagcagtcagaccacaagtggcagtgccatttccacccagcagaacatcgtgggaacagctgaagtgtcttctgccagaacagctccgtcatacccagacacccatgcggtaactgcatacctgcagaagctgtcagagcattcgatgagggagaaccatgagcctggagaaaagccaccccaggtccagcctcttgtagaagaagcagttcctgatcttcacagtccaccaaccacaagcacctcagccctcaacagcttggtggtctcctgtgcatctgctgttggtgtcagagtggctgccacctatgaagctggggccttgtctctcaagaaagttatgaacttttacagcactgccccctgcgagacggcagctcagtcgggcagtgcctccacaggcccagaaagtctgaaagatctccgagaagagcaggtgaaacaggagtcactgcaagggaagaaaagctcaagtctcatggacatcagagaggaagagtcggagggagggagtcgaagactctccctcccagggttgttgtcgcaagtttcccccaggctcttaaggaaggctgcgagggtgaaaactcggacagtggttctgactcccacatacagtggagaagcagatgcccttctgccttccctgaggacagaggtgtggacctgggggaaaggcaaggaagggcagctagggcacggcgacgtcctgcccaggcttcagccgttgtgtgtcaagtgtctggatggtaaagaggtaatccacctggaggcgggcggctcccactccctcgcactcactgcgaaatctcaggtttactcatggggcagtaatacctttggtcagcttgggcattctgagtttccaacaacggttcctcgactctcaaaggttagcagtgaaaatggagtctggagtgtagctgcaggccaagattattccttgtttttagtggacacggaagacttccagcctgggttgtattacagtggccgacaggaccgtgcagaaggtgataccctgccagagaatcccagtggtacaaagactccagtacttctctcctgtagtaagcttggatacataagcagagtaacagcaggaaaagatagctatctagccttggtggataagaacatcatgggatacatcgccagtctccatgagttggcttctacagaaagacggttttactcaaaactgagcgaaatcaaatcacagatacttaggcctcttctcagtttagaaaatttgggcacagtgaccactgtccagctgttgcaggaagttgccagccggttcagcaagttgtgttacctcattgggcagcatggagcctcactaagcagctacctacagggtatgaaggaagccagcagcctggtcatcatgaagcactcaagtcttttcctggacagctacacagagtactgcacatcagtttcaaatttcctggttatgggaggattccagcttcttgctaagcctgccattgatttcctaaataaaaaccaagaactcttgcaagatttgtcagaagtgaatgatgagaacactcagttgatggaaatcctgaacatgctgtttttcttgccaatcagacgacttcataattatgcaaaagttttgctaaagcttgccacttgctttgaagtgacatctccagagtatcaaaagctgcaggattccagttcttgctatgagtctcttgctctccatcttggcaagaagaggaaggaagcagagtacacactgagcttctggaagacctttcctgggaaaatgacggattccttgaggaagccagagcgccggctgctgtgtgagagcagtaaccgagccctctccctgcagcatgccggcaggttttctgtgaattggttcattctcttcaatgatgccctggtccatgctcagttctctacacaccacgtgttccctttggccacactctgggcagagccactatctgaagaagctggtagcgtgaatggcttaaagataactacacctgaagaacaattcacactcatttcttcaacaccccaggaaaagaccaagtggcttcgggctattagccaagctgtggatcaggctttgagggggacgtccgatttcccactttacggaggcggcagcagtgttcagagacaggaaccacccatctcaagaagtgccaaatacactttctacaaggatactcgcctaaaggatgccacttacgatgggcgctggctttcagggaagcctcatggcaggggtgttctgaagtggcctgatggaaagatgtactctggcatgttcaggaatggcttggaagatggatatggtgaatacagaatccctaacaaggccctgaacaaagaagaccattatgtaggccattggaaagaggggaaaatgtgtgggcaaggagtctacagctatgcctctggtgaagtgtttgaaggctgctttcaagataacatgcgccatgggcatggtctgctccggagtggaaaactgacttcttcttctcctagcatgttcattggccagtgggtaatggataagaaagcaggatatggcgtctttgatgatatcaccaggggagaaaagtacatgggaatgtggcaggatgatgtgtgccaagggaatggggtagtagtcacccagtttgggttatactacgaaggcaacttccacctgaataagatgatgggaaatggggttttgctttctgaagatgataccatctatgaaggagaattttccgatgactggacacttagtggaaagggaacgctgactatgccacatggagattatattgaaggttattttagtggagaatggggatctgggataaaaatcactgggacctacttcaaacctagcctgtatgagagcgataaggacaagcccaaagccttcaggaagctggggaacctggccgtggcagcagacgagaaatggagagcagtgtttgaagaatgctggcaccagctgggctgtgagagcccaggccaaggggaggtttggaaagcatgggataatattgctgtggccttgaccacgaaccgtcgccagcataaagacagtccagaaatactaagccgctctcagactcagaccctggagagtttggagtacattccccagcacattggcgccttctctgtggagaaatatgatgacatcaagaagtatttaataaaggcctgtgatactcctctgcacccactgggcaggcttgtggagaccctggttgcggtgtatagaatgacatatgtgggtgtaggggccaaccgccggttactgcaggaagctgtgaaggagattaaatcttatctcaagaggattttccagcttgtgaggttcttgtttcctgagcttcctgaggagggcagcacaattcctctttctgctcctctgcccactggaaggagatccttctgtactgggaaattggattccagatccgagtcaccagaaccaggttatgtagtaacaagttctggcttactgcttccggtgctgctgccgcggctctacccacctctcttcatgctctatgccctggataatgaccgagaggaagacatttactgggaatgtgtgcttcgactaaacaagcagccagatattgctctcttgggcttccttggagtacagaaaaaattctggccagccaccttgtcaatccttggagagagtaaaaaggtgttgtcaaccacaaaggatgcttgctttgcatctgcagtagaatgcctgcagcagatcagcacaacatttactccatcagacaagcttaaagtgatccagcagacctttgaagagatctcccagagtgtccttgcatcgctgcaggaggacttcctctggtccatggatgacttgttccccgtcttcttatacgtggtgctgcgggccaggattcggaacttgggctctgaagttcacctcattgaggatctgatggacccctttctccagcatggggaacaaggcatcatgttcaccaccttgaaggcctgttacttccagattcagcgggagaagcttaactagggcgcctgacagcttgaggaccggattatctgctgcggaggctacagctatggcacaggcaccgactggaggctgatggggcaaagaacagtgttgaatacagaatggacttttgtgctattttggttgtaatttctgagccttactaataattagagcccagcatggaaaacatactgtatcattcaaatggagactggaaaaggagatagggatagagtagagtctttggcctgtgctgagatccacacacctacttagaaaaggaaactggttaccctttcctgtagtgaaagctctcagctccatgcagttccaggaaacctttccaggaaagctgcttagatgaaaagaagttgatgactgtgtttaagctcctggtttgtctaattccatttgcagttacccaataccctttggcaaggagcaggttttacttgaaactgaagcagccatcccttgccttcctagacctctcgctcccaggcacaagtgcagcatgctactttgctaggggtgggggtgggggagaagaagttttaaactgtagttttaaccttttgtaagcccctttaccaaggcatttgtggtcagagagctcccacggggtgactatgacatcctggtcccctcgtggaatgcatccacataggatcttctgcctgctgactgaaaagaacataggaatacactggagtgcaaacactgccgtgccaagctgctccaaacctcactgatccgaggcccactgcctacccaggaggcccactgcctacccaggaggcccgtaagcttcttagcacaagctttgtgtggagactgaagatctgcacatgtgaggaagcagggagctacagtggccctcagcccagtctgcgggtcttccctctacctcacactgaactcagaagggaaggaaggagagacgcacatgggattctcccacctcagaagtattgtgacagcaccgcataaccacggtttgctcttttacaagcagcctcacaagtgtgggttgtgggtgtgcgctggagcagtgccactcgtagctgtttggataccacaggtgctcttccgtctcatctgctgtactcggaggcgagcgcagtggcctgactcatgggaaatgactcagcaggcggcaactacttttgaaaaggatcatgatttccgagctactttataatcaagacaagcatttgttaacatactgttttggaatgttggctgtaatgtaacagcagttttcataataaatgacattcatctc

SEQ ID NO: 5 - protein, 1651 aa, Mus musculus
MDSKKKSSTEAEGSKERGLVHVWQAGSFSLTPERLPGWGGKTVLQAALGVRHGVLLTEDGEVYSFGTLPWKSESAEICPSSPLLESALVGHHVITVATGSFHSGAVTESGVVYMWGENAAGQCAVANQQYVPEPSPVSISDSETSPSLAVRILQLACGEEHTLALSLSREIWAWGTGCQLGLITTTFPVTKPQKVEHLAGRVVLQVACGAFHSLALVQCLPPQDLKPVPERCNQCSQLLITMTDKEDHVIISDSHCCPLGVTLSESQAEKHASPAPSPHPEALDEQGEVFENTVVEAELNMGSSQTTSGSAISTQQNIVGTAEVSSARTAPSYPDTHAVTAYLQKLSEHSMRENHEPGEKPPQVQPLVEEAVPDLHSPPTTSTSALNSLVVSCASAVGVRVAATYEAGALSLKKVMNFYSTAPCETAAQSGSASTGPESLKDLREEQVKQESLQGKKSSSLMDIREEESEGGSRRLSLPGLLSQVSPRLLRKAARVKTRTVVLTPTYSGEADALLPSLRTEVWTWGKGKEGQLGHGDVLPRLQPLCVKCLDGKEVIHLEAGGSHSLALTAKSQVYSWGSNTFGQLGHSEFPTTVPRLSKVSSENGVWSVAAGQDYSLFLVDTEDFQPGLYYSGRQDRAEGDTLPENPSGTKTPVLLSCSKLGYISRVTAGKDSYLALVDKNIMGYIASLHELASTERRFYSKLSEIKSQILRPLLSLENLGTVTTVQLLQEVASRFSKLCYLIGQHGASLSSYLQGMKEASSLVIMKHSSLFLDSYTEYCTSVSNFLVMGGFQLLAKPAIDFLNKNQELLQDLSEVNDENTQLMEILNMLFFLPIRRLHNYAKVLLKLATCFEVTSPEYQKLQDSSSCYESLALHLGKKRKEAEYTLSFWKTFPGKMTDSLRKPERRLLCESSNRALSLQHAGRFSVNWFILFNDALVHAQFSTHHVFPLATLWAEPLSEEAGSVNGLKITTPEEQFTLISSTPQEKTKWLRAISQAVDQALRGTSDFPLYGGGSSVQRQEPPISRSAKYTFYKDTRLKDATYDGRWLSGKPHGRGVLKWPDGKMYSGMFRNGLEDGYGEYRIPNKALNKEDHYVGHWKEGKMCGQGVYSYASGEVFEGCFQDNMRHGHGLLRSGKLTSSSPSMFIGQWVMDKKAGYGVFDDITRGEKYMGMWQDDVCQGNGVVVTQFGLYYEGNFHLNKMMGNGVLLSEDDTIYEGEFSDDWTLSGKGTLTMPHGDYIEGYFSGEWGSGIKITGTYFKPSLYESDKDKPKAFRKLGNLAVAADEKWRAVFEECWHQLGCESPGQGEVWKAWDNIAVALTTNRRQHKDSPEILSRSQTQTLESLEYIPQHIGAFSVEKYDDIKKYLIKACDTPLHPLGRLVETLVAVYRMTYVGVGANRRLLQEAVKEIKSYLKRIFQLVRFLFPELPEEGSTIPLSAPLPTGRRSFCTGKLDSRSESPEPGYVVTSSGLLLPVLLPRLYPPLFMLYALDNDREEDIYWECVLRLNKQPDIALLGFLGVQKKFWPATLSILGESKKVLSTTKDACFASAVECLQQISTTFTPSDKLKVIQQTFEEISQSVLASLQEDFLWSMDDLFPVFLYVVLRARIRNLGSEVHLIEDLMDPFLQHGEQGIMFTTLKACYFQIQREKLN

SEQ ID NO: 6 - DNA, 20 bp, Artificial Sequence
  Artificial Sequence Synthesized Oligonucleotide
cctagtcatccatgtgctgg

SEQ ID NO: 7 - DNA, 20 bp, Artificial Sequence
  Artificial Sequence Synthesized Oligonucleotide
tcccatacctgaccttccac

SEQ ID NO: 8 - DNA, 24 bp, Artificial Sequence
  Artificial Sequence Synthesized Oligonucleotide
cttgatagactttctgtaaagaag

SEQ ID NO: 9 - DNA, 24 bp, Artificial Sequence
  Artificial Sequence Synthesized Oligonucleotide
ggctacttggacaaatctccactg

SEQ ID NO: 10 - DNA, 20 bp, Artificial Sequence
  Description of Artificial Sequence synthesized oligonucleotide
ggagagactgtgctcccaag

SEQ ID NO: 11 - DNA, 20 bp, Artificial Sequence
  Description of Artificial Sequence synthesized oligonucleotide
agccctccttagccaatagc

SEQ ID NO: 12 - DNA, 20 bp, Artificial Sequence
  Description of Artificial Sequence synthesized oligonucleotide
taagcttagtgggcaggctc

SEQ ID NO: 13 - DNA, 22 bp, Artificial Sequence
  Description of Artificial Sequence synthesized oligonucleotide
ttcccacttaacaaccatcaac

SEQ ID NO: 14 - DNA, 24 bp, Artificial Sequence
  Description of Artificial Sequence synthesized oligonucleotide
ccaatttggttaaatctatagggg

SEQ ID NO: 15 - DNA, 20 bp, Artificial Sequence
  Description of Artificial Sequence synthesized oligonucleotide
gacaatgccagagtgtgctc

SEQ ID NO: 16 - DNA, 20 bp, Artificial Sequence
  Description of Artificial Sequence synthesized oligonucleotide
ccagccctttgttagcagtc

SEQ ID NO: 17 - DNA, 20 bp, Artificial Sequence
  Description of Artificial Sequence synthesized oligonucleotide
cttcttcctgcctgtcaagg

SEQ ID NO: 18 - DNA, 20 bp, Artificial Sequence
  Description of Artificial Sequence synthesized oligonucleotide
ttgtacaatgcctcccttcc

SEQ ID NO: 19 - DNA, 20 bp, Artificial Sequence
  Description of Artificial Sequence synthesized oligonucleotide
agcccaacatgacacctttc

SEQ ID NO: 20 - DNA, 20 bp, Artificial Sequence
  Description of Artificial Sequence synthesized oligonucleotide
gattgcttgttgcataaggg

SEQ ID NO: 21 - DNA, 20 bp, Artificial Sequence
  Description of Artificial Sequence synthesized oligonucleotide
atacagcatgcgatgtcagg

SEQ ID NO: 22 - DNA, 20 bp, Artificial Sequence
  Description of Artificial Sequence synthesized oligonucleotide
ctggactcccactccttcac

SEQ ID NO: 23 - DNA, 21 bp, Artificial Sequence
  Description of Artificial Sequence synthesized oligonucleotide
gctagaagagcccagatttcc

SEQ ID NO: 24 - DNA, 20 bp, Artificial Sequence
  Description of Artificial Sequence synthesized oligonucleotide
tgactttgtgtgcctgtgtg

SEQ ID NO: 25 - DNA, 20 bp, Artificial Sequence
  Description of Artificial Sequence synthesized oligonucleotide
ataccctggaaaatctgggg

SEQ ID NO: 26 - DNA, 20 bp, Artificial Sequence
  Description of Artificial Sequence synthesized oligonucleotide
tttgcgcattatctctggtc

SEQ ID NO: 27 - DNA, 20 bp, Artificial Sequence
  Description of Artificial Sequence synthesized oligonucleotide
gtacgtatgaaattcccccg

SEQ ID NO: 28 - DNA, 20 bp, Artificial Sequence
  Description of Artificial Sequence synthesized oligonucleotide
ttccgtcttactcctgcacc

SEQ ID NO: 29 - DNA, 20 bp, Artificial Sequence
  Description of Artificial Sequence synthesized oligonucleotide
gccttaggatccaattcctg

SEQ ID NO: 30 - DNA, 23 bp, Artificial Sequence
  Description of Artificial Sequence synthesized oligonucleotide
caatgatgtactgatgaaccagc

SEQ ID NO: 31 - DNA, 20 bp, Artificial Sequence
  Description of Artificial Sequence synthesized oligonucleotide
cctgatggtttaatggtggg

SEQ ID NO: 32 - DNA, 20 bp, Artificial Sequence
  Description of Artificial Sequence synthesized oligonucleotide
gcacatggcaacaggttaag

SEQ ID NO: 33 - DNA, 20 bp, Artificial Sequence
  Description of Artificial Sequence synthesized oligonucleotide
tccttggcagaataaccctg

SEQ ID NO: 34 - DNA, 20 bp, Artificial Sequence
  Description of Artificial Sequence synthesized oligonucleotide
cccctaccactccctttacc

SEQ ID NO: 35 - DNA, 23 bp, Artificial Sequence
  Description of Artificial Sequence synthesized oligonucleotide
ccagtggctaatagtacctgtcc

SEQ ID NO: 36 - DNA, 20 bp, Artificial Sequence
  Description of Artificial Sequence synthesized oligonucleotide
tggatgcatgattcatttcc

SEQ ID NO: 37 - DNA, 20 bp, Artificial Sequence
  Description of Artificial Sequence synthesized oligonucleotide
tccttggctttccaaatgtc

SEQ ID NO: 38 - DNA, 20 bp, Artificial Sequence
  Description of Artificial Sequence synthesized oligonucleotide
ctatcctggggtctctgctg

SEQ ID NO: 39 - DNA, 20 bp, Artificial Sequence
  Description of Artificial Sequence synthesized oligonucleotide
tgctatcgaaatggttgctg

SEQ ID NO: 40 - DNA, 20 bp, Artificial Sequence
  Description of Artificial Sequence synthesized oligonucleotide
agctacgaccagcaaattcc

SEQ ID NO: 41 - DNA, 20 bp, Artificial Sequence
  Description of Artificial Sequence synthesized oligonucleotide
ataggggtccacctttcagg

SEQ ID NO: 42 - DNA, 20 bp, Artificial Sequence
  Description of Artificial Sequence synthesized oligonucleotide
aaggggatatgggcagagtc

SEQ ID NO: 43 - DNA, 20 bp, Artificial Sequence
  Description of Artificial Sequence synthesized oligonucleotide
aaatgcttgcttggttttgg

SEQ ID NO: 44 - DNA, 20 bp, Artificial Sequence
  Description of Artificial Sequence synthesized oligonucleotide
aaagggcatcttcattgcac

SEQ ID NO: 45 - DNA, 20 bp, Artificial Sequence
  Description of Artificial Sequence synthesized oligonucleotide
cacaagaggcagaaagagcc

SEQ ID NO: 46 - DNA, 21 bp, Artificial Sequence
  Description of Artificial Sequence synthesized oligonucleotide
aatgcttgatgaattgttgcc

SEQ ID NO: 47 - DNA, 20 bp, Artificial Sequence
  Description of Artificial Sequence synthesized oligonucleotide
atgatcatcctcaccccagg

SEQ ID NO: 48 - DNA, 20 bp, Artificial Sequence
  Description of Artificial Sequence synthesized oligonucleotide
ttgaagatttatgcctgggg

SEQ ID NO: 49 - DNA, 20 bp, Artificial Sequence
  Description of Artificial Sequence synthesized oligonucleotide
tgaggtcacacggctatcag

SEQ ID NO: 50 - DNA, 20 bp, Artificial Sequence
  Description of Artificial Sequence synthesized oligonucleotide
gtgtagtggggctgatgtcc

SEQ ID NO: 51 - DNA, 20 bp, Artificial Sequence
  Description of Artificial Sequence synthesized oligonucleotide
tggctatgcaaacattcagg

SEQ ID NO: 52 - DNA, 21 bp, Artificial Sequence
  Description of Artificial Sequence synthesized oligonucleotide
aatgcaaaataccacacatgg

SEQ ID NO: 53 - DNA, 20 bp, Artificial Sequence
  Description of Artificial Sequence synthesized oligonucleotide
tcattggcttaaactgtggg

SEQ ID NO: 54 - DNA, 20 bp, Artificial Sequence
  Description of Artificial Sequence synthesized oligonucleotide
caacctagggttgatgcctg

SEQ ID NO: 55 - DNA, 20 bp, Artificial Sequence
  Description of Artificial Sequence synthesized oligonucleotide
catcttcggaaagcaaaacc

SEQ ID NO: 56 - DNA, 20 bp, Artificial Sequence
  Description of Artificial Sequence synthesized oligonucleotide
ctttggggatatgactgcgt

SEQ ID NO: 57 - DNA, 26 bp, Artificial Sequence
  Description of Artificial Sequence synthesized oligonucleotide
gtaaaagaatttattagggagaaaaa

SEQ ID NO: 58 - DNA, 24 bp, Artificial Sequence
  Description of Artificial Sequence synthesized oligonucleotide
ttcctctaaccccacattttattc

SEQ ID NO: 59 - DNA, 26 bp, Artificial Sequence
  Description of Artificial Sequence synthesized oligonucleotide
tgcttttaaaatattaaccagctttg

SEQ ID NO: 60 - DNA, 21 bp, Artificial Sequence
  Description of Artificial Sequence synthesized oligonucleotide
tcagtcttggcagttttggtc

SEQ ID NO: 61 - DNA, 21 bp, Artificial Sequence
  Description of Artificial Sequence synthesized oligonucleotide
ctgctgtatgttgagcaggtg

SEQ ID NO: 62 - DNA, 20 bp, Artificial Sequence
  Description of Artificial Sequence synthesized oligonucleotide
tggatgctccactttgactg

SEQ ID NO: 63 - DNA, 20 bp, Artificial Sequence
  Description of Artificial Sequence synthesized oligonucleotide
ttaagaacccccttgagtgc

SEQ ID NO: 64 - DNA, 20 bp, Artificial Sequence
  Description of Artificial Sequence synthesized oligonucleotide
ttcctggtcccaaaattgac

SEQ ID NO: 65 - DNA, 20 bp, Artificial Sequence
  Description of Artificial Sequence synthesized oligonucleotide
cagggtgaaactacccaagc

SEQ ID NO: 66 - DNA, 20 bp, Artificial Sequence
  Description of Artificial Sequence synthesized oligonucleotide
ttttatgcttttcaaccccc

SEQ ID NO: 67 - DNA, 20 bp, Artificial Sequence
  Description of Artificial Sequence synthesized oligonucleotide
acacactttctcgctgggac

SEQ ID NO: 68 - DNA, 20 bp, Artificial Sequence
  Description of Artificial Sequence synthesized oligonucleotide
tgatctgagcacaaaggctg

SEQ ID NO: 69 - DNA, 20 bp, Artificial Sequence
  Description of Artificial Sequence synthesized oligonucleotide
taaacagcggtgggtagagc

SEQ ID NO: 70 - DNA, 21 bp, Artificial Sequence
  Description of Artificial Sequence synthesized oligonucleotide
aatgctccttttctcccactc

SEQ ID NO: 71 - DNA, 20 bp, Artificial Sequence
  Description of Artificial Sequence synthesized oligonucleotide
tgccaaatttccaataatgc

SEQ ID NO: 72 - DNA, 19 bp, Artificial Sequence
  Description of Artificial Sequence synthesized oligonucleotide
taatggggacaaggaagcc

SEQ ID NO: 73 - DNA, 20 bp, Artificial Sequence
  Description of Artificial Sequence synthesized oligonucleotide
gctgaggcaaaacaagcatc

SEQ ID NO: 74 - DNA, 20 bp, Artificial Sequence
  Description of Artificial Sequence synthesized oligonucleotide
ccaaagacctgcactctgac

SEQ ID NO: 75 - DNA, 20 bp, Artificial Sequence
  Description of Artificial Sequence synthesized oligonucleotide
ctggcttggctctctcctac

SEQ ID NO: 76 - DNA, 20 bp, Artificial Sequence
  Description of Artificial Sequence synthesized oligonucleotide
aaaaagcacgatcaaatggc

SEQ ID NO: 77 - DNA, 20 bp, Artificial Sequence
  Description of Artificial Sequence synthesized oligonucleotide
ggaagagcgtactcctgctg

SEQ ID NO: 78 - DNA, 20 bp, Artificial Sequence
  Description of Artificial Sequence synthesized oligonucleotide
gcaggagtacgctcttccac

SEQ ID NO: 79 - DNA, 22 bp, Artificial Sequence
  Description of Artificial Sequence synthesized oligonucleotide
gaacaaaatgtgctctaaaggc

SEQ ID NO: 80 - DNA, 20 bp, Artificial Sequence
  Description of Artificial Sequence synthesized oligonucleotide
tctttttctctctggggcag

SEQ ID NO: 81 - DNA, 21 bp, Artificial Sequence
  Description of Artificial Sequence synthesized oligonucleotide
tgccttctgtgttttaccctg

SEQ ID NO: 82 - DNA, 20 bp, Artificial Sequence
  Description of Artificial Sequence synthesized oligonucleotide
gaagggaacagggaaaagtg

SEQ ID NO: 83 - DNA, 21 bp, Artificial Sequence
  Description of Artificial Sequence synthesized oligonucleotide
ttacctccctttcaatcctcc

SEQ ID NO: 84 - protein, 545 aa, Homo sapiens
MDSKKRSSTEAEGSKERGLVHIWQAGSFPITPERLPGWGGKTVLQAALGVKHGVLLTEDGEVYSFGTLPWRSGPVEICPSSPILENALVGQYVITVATGSFHSGAVTDNGVAYMWGENSAGQCAVANQQYVPEPNPVSIADSEASPLLAVRILQLACGEEHTLALSISREIWAWGTGCQLGLITTAFPVTKPQKVEHLAGRVVLQVACGAFHSLALVQCLPSQDLKPVPERCNQCSQLLITMTDKEDHVIISDSHCCPLGVTLTESQAENHASTALSPSTETLDRQEEVFENTLVANDQSVATELNAVSAQITSSDAMSSQQNVMGTTEISSARNIPSYPDTQAVNEYLRKLSDHSVREDSEHGEKPMPSQPLLEEAIPNLHSPPTTSTSALNSLVVSCASAVGVRVAATYEAGALSLKKVMNFYSTTPCETGAQAGSSAIGPEGLKDSREEQVKQESMQGKKSSSLVDIREEETGRQSKTLPPWIVVTSFPQALKKGCTGENEDSGSDPHIQWRSRCAPAFSENRSVDLGEREGRAAGARRCSA

(19) United States
(12) Patent Application Publication
(10) Pub. No.: US 2004/0137450 A1
(43) Pub. Date: Jul. 15, 2004

(54) Als2 gene and amyotrophic lateral sclerosis type 2

(76) Inventors: Hadano Shinji, Kanagawa (JP); Joh-E lkeda, Kanagawa (JP); Michael R Hayden, Vancouver (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/467,909

(22) PCT Filed: Feb. 12, 2002
(86) PCT No.: PCT/CA02/00147

(30) Foreign Application Priority Data
Apr. 16, 2001 (JP) 2001-116973

(51) Int. Cl. C12Q 1/68; A01K 67/027; C07H 21/04; C07K 14/47

(52) U.S. Cl. 435/6; 435/69.1; 435/320.1; 435/325; 530/350; 514/12; 800/14; 536/23.5

(57) ABSTRACT

A human gene associated with amyotrophic lateral sclerosis type 2(ALS2) is provided. The gene is present in the second chromosome q33 region. Also provided are mutant versions of the gene as well as isolated nucleic acids derived from the gene and peptides encoded thereby. Methods for the diagnosis of ALS2 are alos provided.

### RELATED APPLICATIONS

This application claims priority from U.S. application No. 60/267,723 filed Feb. 12, 2001; Japanese application no. 2001-116973 filed Apr. 16, 2001; and, U.S. application No. 60/318,352 filed Sep. 12, 2001, which applications are hereby incorporated by reference.

### FIELD OF THE INVENTION

This invention relates to genetic causes of amyotrophic lateral sclerosis of type 2 (“ALS2”).

### BACKGROUND OF THE INVENTION

Amyotrophic lateral sclerosis (“ALS”) is a progressive neurodegenerative disease in which distal and proximal motor neurons are selectively degenerated¹. Its cause is ambiguous and its onset is mostly at middle age and thereafter. Its rate of onset is about 2-6 per 100,000 persons and begins with lowering of muscular strength and myoatrophy of wrist muscle as a secondary neuron hindrance resulting in bulbar paralysis symptoms such as atrophy of muscle of limbs, atrophy of tongue, alalia, dysphagia and dyspnea. No therapeutic method has been established yet and most of the afflicted die within five years from onset.

Juvenile amyotrophic lateral sclerosis of type 2 (“ALS2”; OMIM215100²) is a somatically recessive type hereditary disease. Although the frequency of its onset is rare, muscular convulsion of limbs, face and throat is gradually expressed in persons of teens or twenties and becomes chronic by bulbar paralysis as described above.

Amyotrophic lateral sclerosis of type 2 has been mapped to the 1.7 cM interval flanked by D2S116 and D2S2237 on human chromosome 2q33^{3,4}. Alterations in 391 exons and their flanking regions derived from 43 non-overlapping transcripts have been noted within this interval^{5,6}.

ALS is a very severe disease and there is a need for development of means for its early detection or diagnosis and for treatment.

### SUMMARY OF THE INVENTION

We have now identified a gene associated with amyotrophic lateral sclerosis type 2, termed the ALS2 or ALS2CR6 gene. This gene is expressed in various human tissues including neurons in the brain and spinal cord, and encodes a protein with homology to RanGED and RhoGEF.

This invention now provides mammalian ALS2 genes and mutant versions thereof as well as peptides (including proteins) encoded by such genes. Also included are fragments and nucleic acids derived from these genes, corresponding peptides, and oligonucleotides suitable for use as amplification primers and/or probes. Antibodies to the peptides of this invention are also provided.

This invention also provides methods of diagnosis of ALS2 which may include identifying in a patient at risk, an altered ALS2 gene or protein. The patient may be tested to characterize one or more mutations in the gene or protein produced. Such a mutation may comprise the A261del mutation or the AG1548del mutations described herein.

This invention also provides nucleic acids which correspond to a region of the ALS2 gene, which nucleic acids typically hybridize to at least about 6, at least about 10, at least about 15, at least about 20, or at least about 25 consecutive nucleotides of an ALS2 sequence as described herein, or to complements of such sequences, or to naturally occurring mutants or allelic variants thereof. The probes or primers may be chosen to be capable of distinguishing (such as by amplification or hybridization) allelic variants, including the A261del and AG1548del mutations described herein. Such probes or primers may further include a label which is capable of being detected. This invention also provides kits for identifying ALS2 genes, including those comprising alleles associated with an ALS2 disease state, wherein the kits may comprise a probe or primer as described herein. The kit may further comprise instructions for using the probes or primers to distinguish alleles as described herein.

This invention also provides vectors containing nucleic acids of this invention, including vectors adapted for expression of such nucleic acids in a target cell or organism. Such vectors may comprise appropriate transcription regulatory elements for directing transcription of the nucleic acids in a target cell or organism. Nucleic acids and peptides of this invention may be expressed in bacterial as well as eukaryotic cells, including mammalian cells. Such vectors may be adapted to express nucleic acids of this invention in a reverse direction so as to generate anti-sense transcription products.

This invention also provides non-human mammals comprising a genome in which an ALS2 gene has been mutated, including by deletion. Such a mammal may be a mouse and methods for altering the murine genome such as to produce an ALS2 “knock-out” mouse, are described herein and are known in the art.

This invention also provides the use of nucleic acids and peptides as disclosed herein for the preparation of medicaments for treatment of ALS2 or in the treatment of ALS2.

This invention also provides methods of treating patients for ALS2, which methods may comprise testing the patient to diagnose or characterize an ALS2 disease state. A patient may be treated for ALS2, for example by administering to the patient or by otherwise providing a native form or functional fragment or derivative of the ALS2 peptide described herein or such other therapeutic agent as which will restore function of the protein in a patient. Also included in this invention are vectors suitable for use in gene therapy and gene therapy methodologies whereby a patient is treated to restore the function of ALS2 by delivering or producing a functional gene for expression in the patient. Gene therapy vectors may, for example, be adeno-associated vector, such as those known in the art. General methods for gene therapy are also known in the art.

This invention includes a human ALS2 gene which is present in human second chromosome q33 region and may code for a GTPase regulatory factor. The gene may encode an amino acid sequence of SEQ ID NO:2. cDNA synthesized from mRNA that may be transcribed by this gene has a base sequence of SEQ ID NO:1.

This invention includes a human ALS2 mutated gene which is related to amyotrophic lateral sclerosis of type 2 and codes for a modified protein having an amino acid sequence of SEQ ID NO: 3 or SEQ ID NO:84, by a deficiency of one or two bases of the above human ALS2 gene.

This invention includes nucleic acids purified from genomic DNA, mRNA or cDNA as well as synthesized nucleic acids.

This invention includes oligonucleotides which hybridize to ALS2 genes and variants thereof, preferably under stringent conditions.

This invention includes kits comprising oligonucleotides or oligonucleotide primer sets which may be used to carry out amplification of ALS2 encoded nucleic acids, for example by the polymerase chain reaction (PCR).

This invention includes oligonucleotide probes which hybridize to the regions containing base deficient sites (A261del and AG1548del) in ALS2 under stringent conditions.

This invention includes oligonucleotide primer sets which carry out a PCR amplification of the region containing a base deficient site in ALS2 as described herein. A specific example of this primer set is a pair of synthetic oligonucleotides comprising the base sequences of SEQ ID NO: 6 and NO: 7 or a pair of synthetic oligonucleotides comprising the base sequences of SEQ ID NO: 8 and NO: 9.

This invention includes recombinant vectors comprising the above nucleic acids and cells transcribed by said recombinant vectors.

This invention includes a GTPase regulatory factor or a GEF which is characterized in being an expression product of an ALS2 gene as described herein.

Embodiments of such GTPase regulatory factors of GEF's are recombinant proteins produced by the transformed cells transformed according to this invention.

This invention includes a peptide comprising an amino acid sequence having continuous 5 or more acid amino residues in the first to the 46th amino acid sequence in SEQ ID NO: 2 and also a peptide comprising an amino acid sequence having continuous 5 or more acid amino residues in the 47th to the 1657th amino acid sequence in SEQ ID NO: 2. These peptides may be used for production of antibodies.

This invention also provides a modified protein which may be an expression product of a mutant human ALS2 gene and which comprises the amino acid sequence of SEQ ID NO: 3. An embodiment of this modified protein is a recombinant protein produced by a transformed cell.

This invention includes an antibody which recognizes peptides (including proteins) as disclosed herein. Embodiments of this antibody are an antibody which is prepared using a peptide according to this invention as an antigen, including a peptide comprising an amino acid sequence having continuous 5 or more acid amino residues in the first to the 46th amino acid sequence in SEQ ID NO: 2 and also an antibody which is prepared using a peptide comprising an amino acid sequence having continuous 5 or more acid amino residues in the 47th to the 1657th amino acid sequence in SEQ ID NO: 2 as an antigen.

This invention furthermore provides methods for the diagnosis of amyotrophic lateral sclerosis of type 2 which is characterized in detecting ALS2 mutated genes. An embodiment of this method for the diagnosis it that genomic DNA of the cells of a person to be diagnosed is subjected to a PCR amplification using a primer set comprising a pair of synthetic oligonucleotides comprising the base sequences of SEQ ID NO: 6 and NO: 7 or a pair of synthetic oligonucleotides comprising the base sequences of SEQ ID NO: 8 and NO: 9, the resulting DNA fragments are treated with a restriction enzyme NarI and the said person where each of the DNA fragments is divided into two fragments is judged to be suffering from amyotrophic lateral sclerosis of type 2.

This invention also provides a method for the diagnosis of amyotrophic lateral sclerosis of type 2 which is characterized in that the transcribed product of an ALS2 gene or mutated gene is detected. In an embodiment of this diagnostic method, the transcribed product is cDNA or mRNA of the gene of an ALS2 mutated gene or the modified protein expressed by the said mutated gene. An embodiment of the case of detection of the modified protein is a method for the detection of the protein where the antibody recognizing the first to the 46th amino acid sequences in SEQ ID NO: 2 reacts but the antibody recognizing the 47th to the 1657th amino acid sequence region in SEQ ID NO: 2 does not react.

Further, this invention provides a mouse ALS2 gene which may have an amino acid sequence of SEQ ID NO:5 as well as nucleic acids derived therefrom including nucleic acids synthesized or purified from genomic DNA, mRNA or cDNA of the mouse gene or a complementary sequence thereof.

This invention also provides a gene-defective non-human mammal such as a rodent, preferably a mouse, where function of an ALS2 gene is substantially deficient. Also provided are tissues of such a mouse.

The human ALS2 gene according to this invention is a genomic gene which has 33 introns and 34 exons, exists in a genomic DNA of 80.3 kb adjacent to a polymorphic DNA marker D2S2309 in human second chromosome q 33 region (refer to FIG. 1) and codes for a human GTPase regulatory factor having an amino acid sequence of SEQ ID NO:2. In this ALS2 gene, its cDNA has a base sequence of SEQ ID NO: 1.

This invention provides an isolated nucleic acid that codes for a peptide having at least about 75, 80, 85, 90, 95, 97 or 100% identity to all of an amino acid sequence selected from the group consisting of: SEQ ID NO:2; SEQ ID NO:3; SEQ ID NO:5; SEQ ID NO:84; and, amino acids 372-1657 of SEQ ID NO:2. Also provided are the peptides encoded by these nucleic acids.

This invention also provides an isolated nucleic acid consisting essentially of a nucleotide sequence having at least about 75, 80, 85, 90, 95, 97 or 100% identity to all of a nucleotide sequence or a complementary sequence thereof, selected from the group consisting of: SEQ ID NO:1; SEQ ID NO:4; nucleotides 124-5094 of SEQ ID NO:1; nucleotides 1225-5094 of SEQ ID NO:1; and, nucleotides 124-5076 of SEQ ID NO:4. Also provided are the peptides encoded by these nucleic acids.

The nucleic acids of this invention may be joined to a second nucleic acid not naturally associated with the nucleic acid of this invention. By not naturally associated, it is meant that the second nucleic acid is not part of an ALS2 gene and is not directly joined to an ALS2 gene in the genome of a mammal.

This invention also provides an oligonucleotide of 6 to 75 nucleotides, wherein the oligonucleotide hybridizes to a nucleic acid of this invention or a complementary sequence thereof, under stringent conditions. An oligonucleotide of this invention may be joined to a label, which is any moiety suitable for detectable labelling of the nucleic acid or for binding of the nucleic acid to a non-nucleic acid moiety.

This invention also provides a peptide consisting essentially of a sequence of at least 5 contiguous amino acids from a sequence selected from the group consisting of: amino acids 1-46 of SEQ ID NO:2; amino acids 47-1657 of SEQ ID NO:2; SEQ.ID NO:3; amino acids 43-49 of SEQ ID NO:3; SEQ ID NO:84; and amino acids 476 to 545 of SEQ ID NO:84. These peptides are useful, for example in raising antibodies of this invention and for investigating the function of the ALS2 protein.

This invention also provides a non-human mammal comprising a mutated gene, wherein the gene but for the mutation would encode a protein having at least about 75, 80, 85, 90, 95, 97 or 100% sequence identity to all of SEQ ID NO:2 or SEQ ID NO:5.

This invention also provides a method for the diagnosis of amyotrophic lateral sclerosis type 2 in a patient, comprising detecting the presence of a mutation in a gene that encodes a protein having at least about 75, 80, 85, 90, 95, 97 or 100% sequence identity to SEQ ID NO:2 in a patient or a biological sample from a patient.

This invention also provides a method for the diagnosis of anyotrophic lateral sclerosis type 2, comprising detecting the presence or absence of a protein having at least about 75, 80, 85, 90, 95, 97 or 100% sequence identity to all of SEQ ID NO:2 in a patient or a biological sample from a patient.

This invention also provides a method for the diagnosis of amyotrophic lateral sclerosis type 2, comprising detecting the presence or absence of a protein having at least about 75, 80, 85, 90, 95, 97 or 100% sequence identity to all of SEQ ID NO:3 or SEQ ID NO:84 in a patient or a biological sample from a patient.

In the diagnostic methods of this invention, sequences may be compared to determine the presence of mutations; oligonucleotides may be used to detect hybridization to nucleic acids of the patient; amplification of nucleic acids of the patient may be performed; proteins of the patient may be contacted with antibodies of this invention; or proteins produced in the patient may be evaluated for the function of ALS2 protein.

This invention also provides a method of treatment of amyotrophic lateral sclerosis type 2, comprising administering a peptide, a nucleic acid, or a pharmaceutical composition comprising the peptide or nucleic acid to a patient in need thereof, wherein the peptide comprises an amino acid sequence having at least about 75, 80, 85, 90, 95, 97 or 100% identity to SEQ ID NO:2 or a fragment thereof, and wherein the nucleic acid codes for said peptide.

This invention also provides a method of treatment of amyotrophic lateral sclerosis type 2, comprising administering a composition to a patient in need thereof, wherein the composition mimics the biological activity of the peptide of SEQ ID NO. 2 or a fragment thereof.

This invention also provides the use of a peptide or a nucleic acid for preparation of a medicament for treatment of amyotrophic lateral sclerosis type 2, wherein the peptide comprises an amino acid sequence having at least about 75, 80, 85, 90, 95, 97 or 100% identity to SEQ ID NO:2 or a fragment thereof, and the nucleic acid codes for said peptide.

In this specification the term “isolated” with reference to a nucleic acid or peptide means that a nucleic acid is separate from the genome of a cell, a peptide is separate from a cell but does not mean that the subject matter has been obtained from a genome or a cell. In some instances, nucleic acids and peptides of this invention may be synthesized using conventional techniques.

Two nucleic acid or protein sequences are considered substantially identical if, when optimally aligned, they share at least about 70% sequence identity. In alternative embodiments, sequence identity may for example be at least 75%, at least 90% or at least 95%. Optimal alignment of sequences for comparisons of identity may be conducted using a variety of algorithms, such as the local homology algorithm of Smith and Waterman,1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence alignment may also be carried out using the BLAST algorithm, described in Altschul et al., 1990, J. Mol. Biol. 215:403-10 (using the published default settings).

Nucleic acid sequences of the invention may in some embodiments be substantially identical, such as substantially identical gene targeting substrates and target sequences. The substantial identity of such sequences may be reflected in percentage of identity when optimally aligned that may for example be greater than 50%, 80% to 100%, at least 80%, at least 90% or at least 95%, which in the case of gene targeting substrates may refer to the identity of a portion of the gene targeting substrate with a portion of the target sequence, wherein the degree of identity may facilitate homologous pairing and recombination and/or repair. An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

It is well known in the art that some modifications and changes can be made in the structure of a polypeptide without substantially altering the biological function of that peptide, to obtain a biologically equivalent polypeptide. In one aspect of the invention, LPL S447X therapeutics may include peptides that differ from a portion of the wild-type LPL sequence by conservative amino acid substitutions. As used herein, the term “conserved amino acid substitutions” refers to the substitution of one amino acid for another at a given location in the peptide, where the substitution can be made without loss of function. In making such changes, substitutions of like amino acid residues can be made, for example, on the basis of relative similarity of side-chain substituents, for example, their size, charge, hydrophobicity, hydrophilicity, and the like, and such substitutions may be assayed for their effect on the function of the peptide by routine testing.

In some embodiments, conserved amino acid substitutions may be made where an amino acid residue is substituted for another having a similar hydrophilicity value (e.g., within a value of plus or minus 2.0), where the following hydrophilicity values are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

### BRIEF DESCRIPTION OF THE DRAWINGS

FIG. 1 is a transcription map of 3 Mb region of human chromosome 2q33 including an ALS2 candidate region. The white open rectangle is between D2S116 to D2S2237^{2,3}. Positions of 7 STS markers, 12 polymorphic DNA markers and 42 independent transcription units are shown. Polarity of 38 transcription units are shown by arrows. The location of the ALS2 gene is designated “ALS2CR6” which term may be used interchangeably for ALS2 below.

FIG. 2 shows a process for the detection of ALS2 associated mutations. “a” is an example of the Tunisian and Kuwaiti ALS2 families. Genotypes of the members constituting a family is shown based on previously reported results^{3,4}. “b” shows the result of sequence determination of mutation (A261del) in genomic DNA of the Tunisian ALS2 family. Patient 10797 is A261del of a homozygotic type and the carrier 10784 is a heterozygotic type. The sequence determination was carried out for PCR products. “c” shows the results of determination of mutation (AG1548del) in the genomic DNA in the Kuwaiti ALS2 family. Sequence of the reverse strand of exon 5 in the region of interest are shown. Individual 18279 is a normal sibling, who is unaffected by ALS2 and carries two normal haplotypes. The box in this sequence indicates the position of the bases deleted in affected members. Individual 18281 is an unaffected parent who carries one disease haplotype. The overlapping normal and mutated sequences are shown. Individual 18275 is affected and the figure shows a homozygous CT deletion in the reverse strand of exon 5. The position of the deleted bases is indicated by the arrow. The corresponding forward sequence and coded normal amino acids and novel amino acids produced by frameshifting are indicated. “d” shows segregation of the A261del mutation in the Tunisian ALS2 family. The presence of the deletion was assayed by the digestion with NarI, which only cuts mutated gene product. For exon-PCR products, the 339 bp fragment representing the normal allele was cleaved into two fragments (225 bp and 113 bp) in the mutant allele. For RT-PCR product, the 302 bp product which represents the normal allele was cleaved into two fragments (195 bp and 106 bp) in the mutant allele.

FIG. 3 shows northern blot analysis of the ALS2 (ALS2CR6) mRNA. In “a”, a northern blot containing 2 μg of poly A⁺ MRNA of many adult human tissues is hybridized with exon 4 of ALS2 cDNA. In the lower drawings, the same blot is hybridized with human β-actin cDNA for confirmation of the property and the comparative load of RNA. In the left, size of the ALS2 transcript is shown. In “b”, northern blot containing 10 μg of total RNA obtained normal whole brain and 20 μg of total RNA obtained from lymphocytes of patients and healthy persons (10788 persons) was hybridized to exon 4 of the ALS2 cDNA. The right panel shows an agarose gel electrophoresis of an RNA sample.

FIG. 4 is a comparison of amino acid sequences in human ALS2CR6 and mouse homolog mALS2CR6. The same residues are shown by frames. There are shown the position of the additional three amino acid residues of the Tunisian mutant protein (starting from the 47th amino acid residue), the position of the 25 amino acid residues (starting from the 372nd residue) of a short variant part of the ALS2 gene and the position of the additional 70 amino acid residues of the Kuwaiti mutant protein (starting from the 476^{th} residue).

FIG. 5 shows an expression of ALS2 MRNA in brain and spinal cord of adult mouse. “a” is an arrow-like whole image of an RNA/RNA in situ hybridization using an antisense ALS2 riboprobe while “b” is a control image using a sense strand probe. Significant expression was noted in neurons of hippocampus and dentate gyrus (c and g), Purkinje cells of cerebellum (d and h), neurons of cerebral cortex (e and i) and cinerea of spinal cord including anterior horn cells (f and j). A scale bar shows a length of 10 μm.

FIG. 6 is a result of an amino acid sequence analysis. “a” is a schematic chart of domains and motifs in normal and mutated ALS2 protein. RCC1 is a regulatory factor for chromosome condensation, DH is a homologous domain to Db1, PH is a pleckstrin-homologous domain, MORN is membrane structure and recognition nexus and VPS9 is a vacuole protein for discrimination of 9 domains. “b” is comparison of amino acid sequences of RCC1 repeat-containing regions for human ALS2 (hALS2CR6), mouse ALS2 (mALS2CR6), human (h) RCC1, human (h) RPGR and mouse (m) RPGR. The amino acid residues shown by open frames are the same. Conserved amino acid residues are abundantly contained as well. Positions of the seven blades corresponding to RCC1 are shown according to the literature³⁰.

FIG. 7 is a chart that compares the wild type human, mouse, and short human variant of the ALS2 proteins and the coding products of the A261del (Tunisian) and AG1548del (Kuwaiti) mutations.

### DETAILED DESCRIPTION OF THE INVENTION

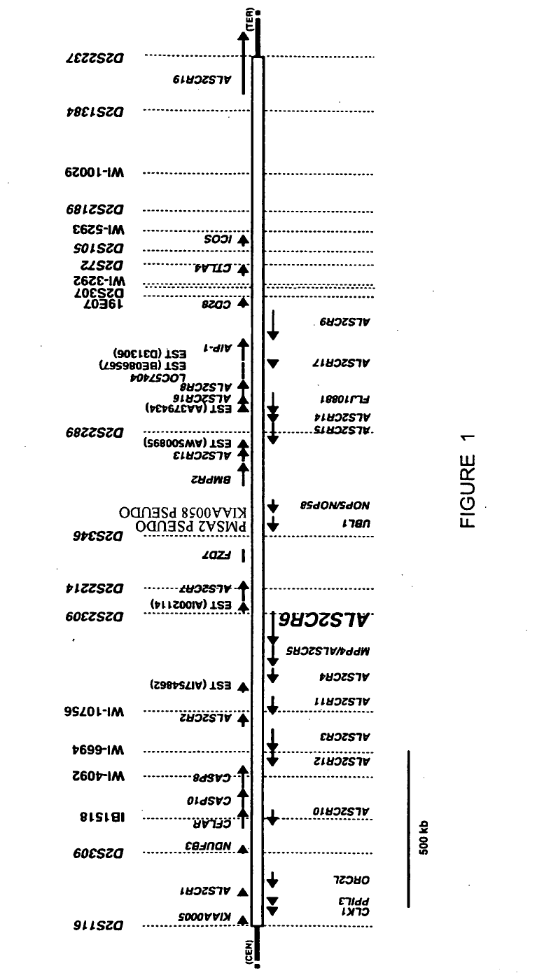

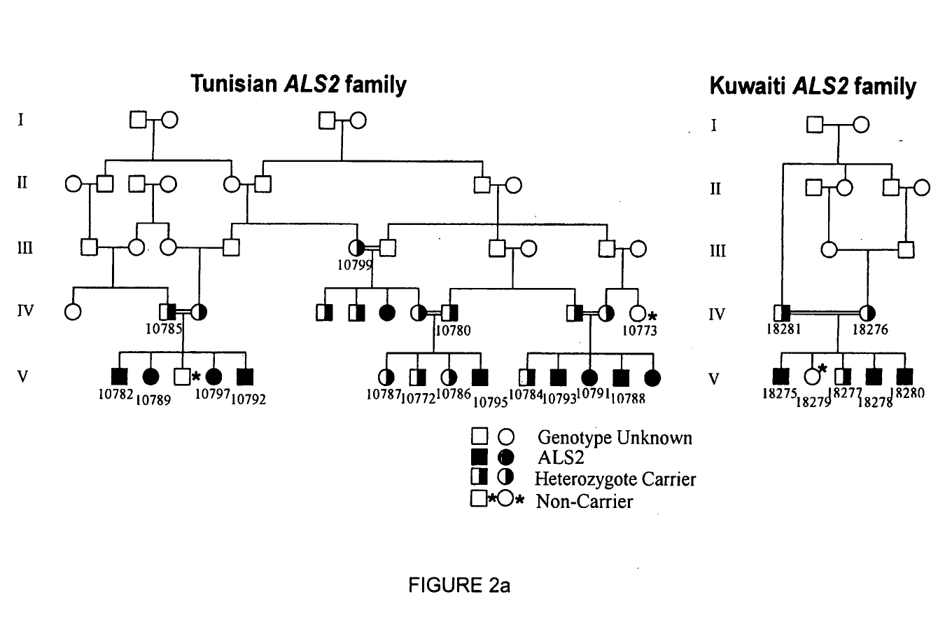

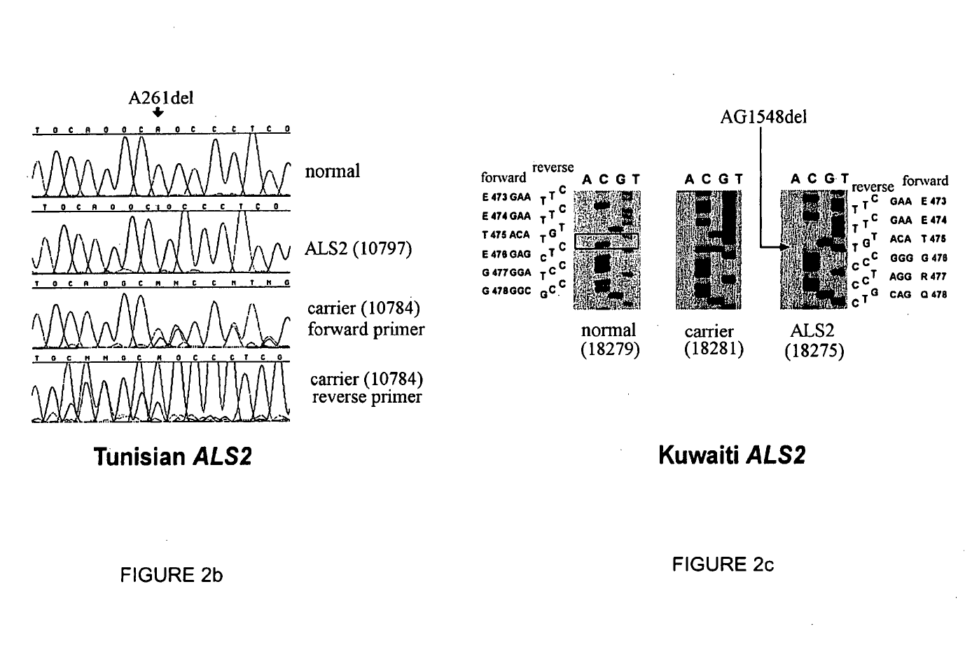

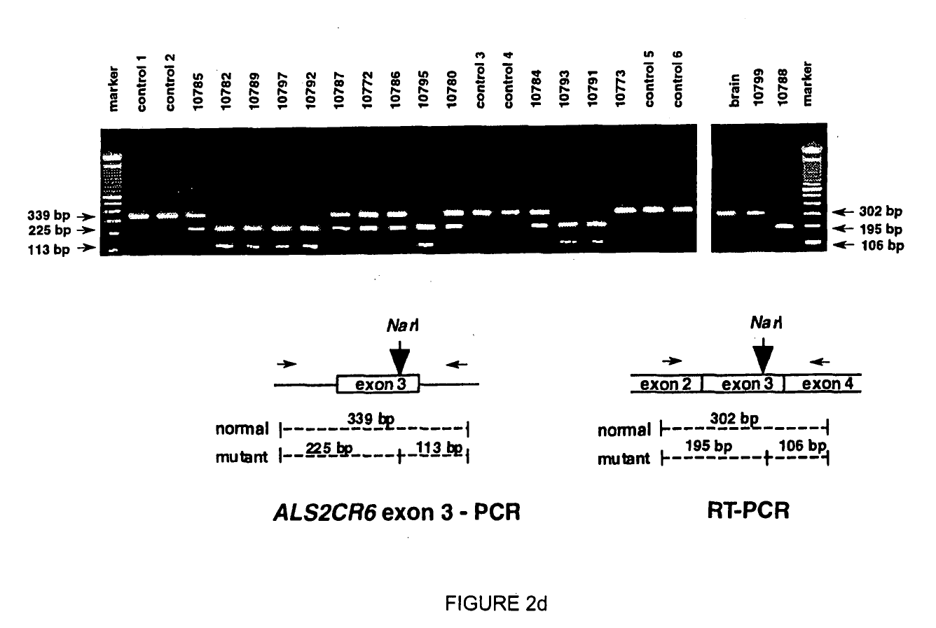

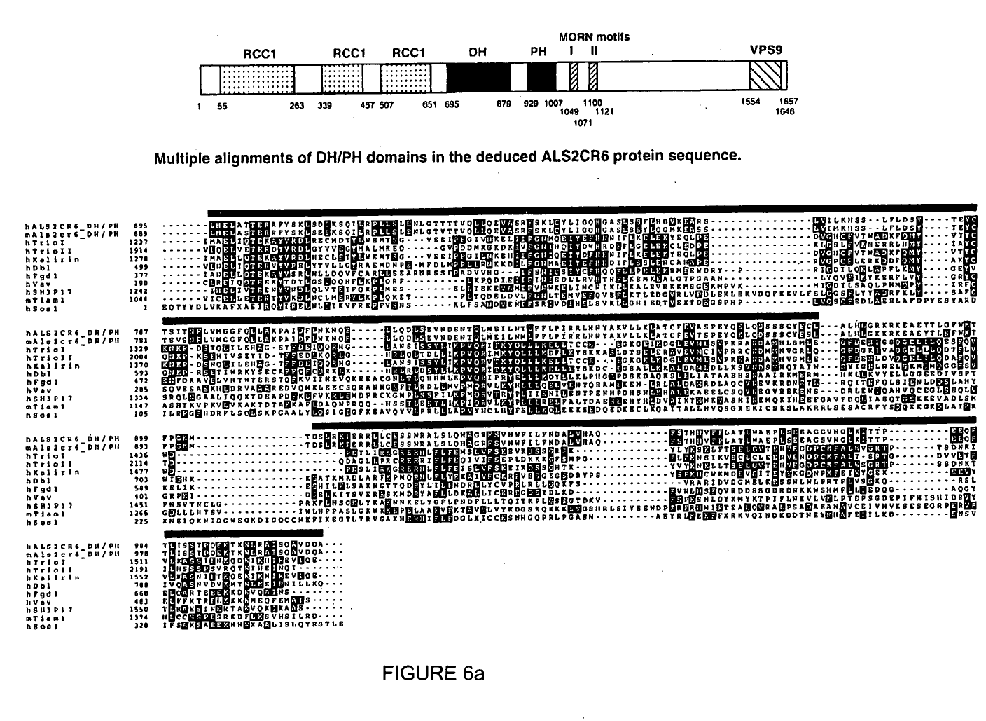

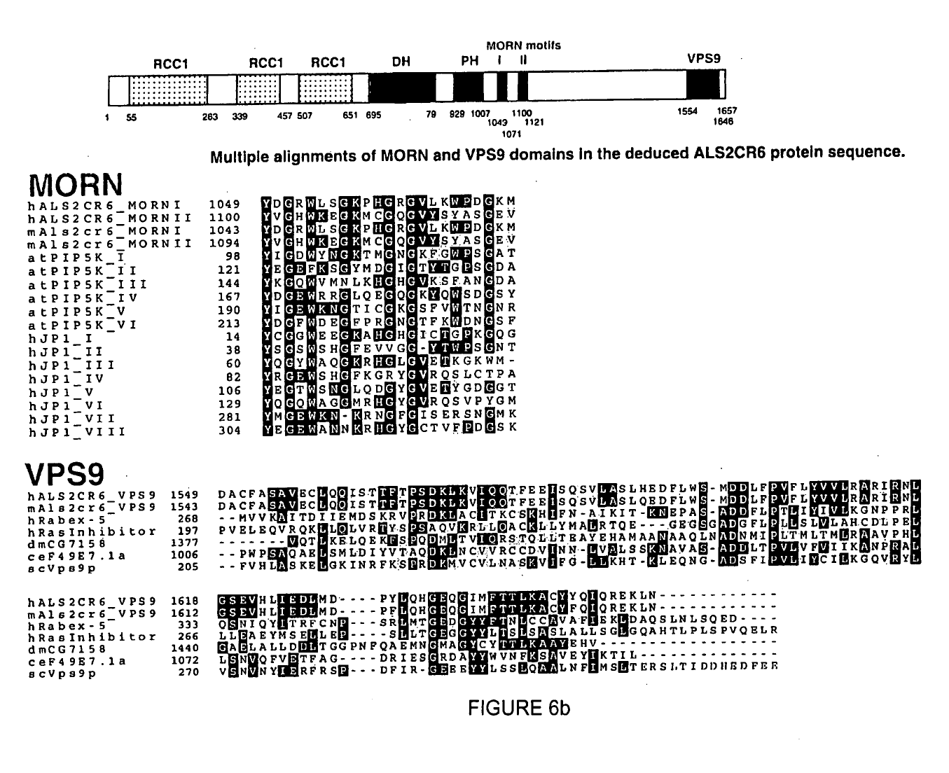

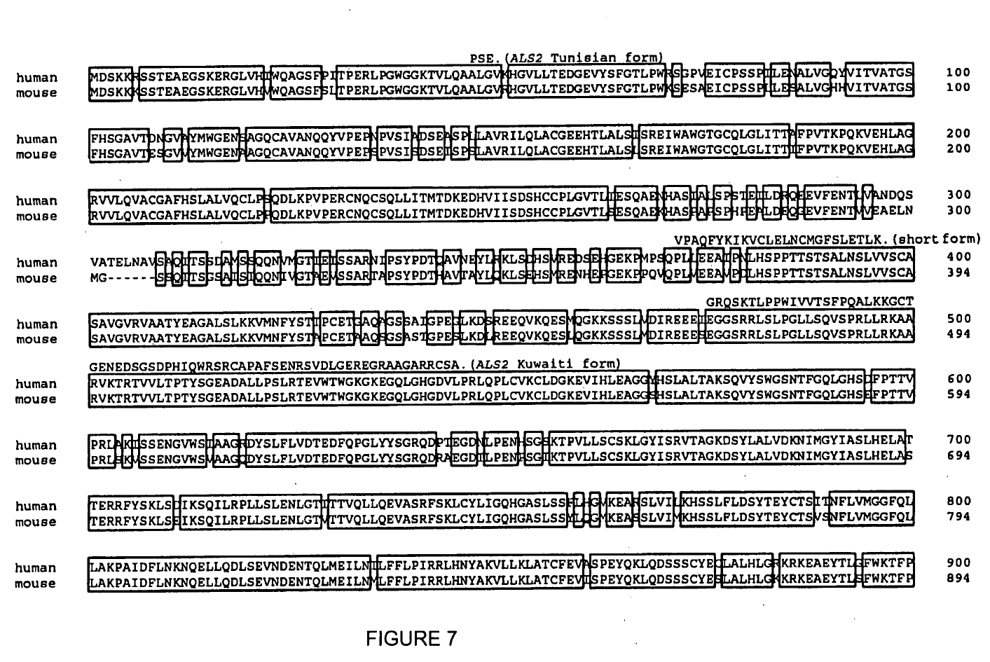

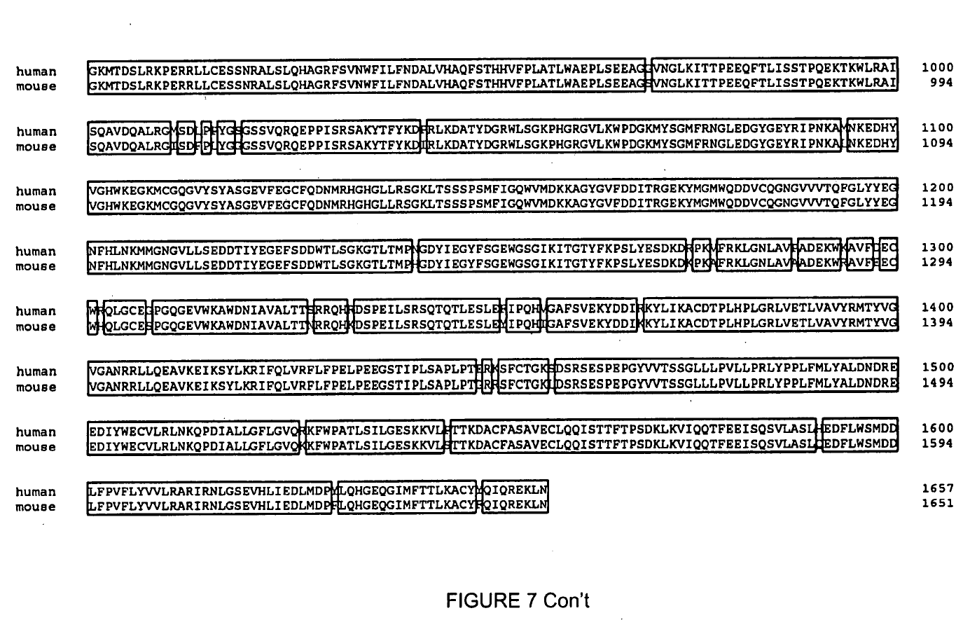

The locus of a 1.7 cM region specified by microsatellite markers D2S116 and D2S2237 of a human second chromosome q 33 region has been mapped^{3,4}. The inventors previously prepared a physical map on the basis of YAC/BAC/PAC of 3Mb genomic region covering the candidate region in FIG. 1^{5,6}. Sequences of cDNA clones and EST's have now been analyzed and 42 non-duplicated transcription units including 10 new genes mapped. 411 pairs in of primers were designed depending upon genomic DNA of 14 persons of a family of ALS2 (FIG. 2a) and 6 normal control persons having no kinship with the former was amplified by PCR. Seventy-seven base sequence polymorphs of introns or exons were identified by determining the sequence for all of the PCR products. Among them, a gene having base deletions related to onset of ALS2 was identified.

The ALS2 gene also includes restriction regions and regulatory regions (promoter/enhancer, suppressor, etc.) which function in expression of protein which is coded thereby. Such restriction and regulation regions are useful for clarifying the functions of the ALS2 gene product as a GEF or a GTPase regulatory factor.

This ALS2 gene may, for example, by isolated by screening a human genome library using pure polynucleotide or oligonucleotide comprising a base sequence of SEQ ID NO: 1 or a partial sequence thereof as a probe. The resulting genomic gene may be amplified by commonly used genetic amplifying methods such as, for example, a PCR (polymerase chain reaction) method, an NASBN (nucleic acid sequence based amplification) method, a TMA (transcription-mediated amplification) method or an SDA (strand displacement amplification) method.

A pure polynucleotide (DNA fragments and RNA fragments) may also be prepared from this ALS2 genomic gene, mRNA transcribed by this gene or cDNA synthesized from mRNA. For example, cDNA may be synthesized using poly(A)+RNA extracted from human cells as a template. The human cells may be either those excised from human body by operation, etc. or incubated cells. cDNA may be synthesized by known methods (Mol. Cell Biol., 2, 161-170, 1982; J. Gene, 25, 263-269, 1983; Gene, 150, 243-250, 1994). One may also synthesize cDNA by an RT-PCR method using an oligonucleotide as a primer and mRNA isolated from human cells as a template. Specifically, the cDNA prepared as such has a base sequence of SEQ ID NO: 1. Those polynucleotides may be used for recombinant expression of a human GTPase regulatory factor.

The oligonucleotides of this invention are DNA fragments or RNA fragments which hybridize to the above-mentioned ALS2 or the above-mentioned nucleic acids under stringent conditions. For example it is a continuous DNA fragment of 10-100 bp in the base sequence of SEQ ID NO: 1. Here, stringent conditions means a condition whereby a specific hybrid formation of target with a probe is made possible by salt concentration, concentration of organic solvent (such as formamide), or temperature condition during hybridization and washing steps. Methods are described in U.S. Pat. No. 6,100,037.

One methodology for creating stringent hybridization conditions is [insert B & K)

A primer set of this invention is typically a pair of oligonucleotides for amplification of ALS2 gene or related nucleic acids. Such a primer set may be designed on the basis of the base sequence of SEQ ID NO: 1, synthesized and subjected to purification using known methods. Size (base numbers) of the primer preferably is 15-40 bases or more preferably, 15-30 bases which specificity anneal with a template DNA. However, when LA (long accurate) PCR is carried out, it is effective to use primers in excess of 30 bases. A pair (two) primers comprising sense strand (5′-terminal side) and antisense strand (3′-terminal side) should not be complementary. In addition, a self-complementary sequence is to be avoided in a primer to prevent the formation of a hairpin structure. Further, in order to ensure a stable bond to a template DNA, the GC content should be about 50% and occurrence of GC-rich or AT-rich regions in a primer should be avoided. Since an annealing temperature is dependent upon Tm (melting temperature), primers having Tm of 55-65° C. are chosen so as to prepare a PCR product having a high specificity. The final concentration of the primer used in PCR should be about 0.1 to about 1 μM. It is possible to use commercially available software for designing a primer including the Oligo™ software [manufactured by National Bioscience Inc. (U.S.A.)] and Genetyx™ software [manufactured by Software Development KK (Japan)].

Mutated ALS2 genes may be obtained by a method where a DNA library prepared from cells of a patient thought to be suffering from ALS2 is screened with a probe which hybridizes to a region containing mutant (e.g. a base deficient site) under a stringent condition. Pure polynucleotide (DNA fragment or RNA fragment) may be obtained from genomic DNA, mRNA or cDNA of an ALS2 mutated gene or a complementary sequence thereof. For example, an ALS2 mutated gene comprises a nucleic acid where the 261st base a of SEQ ID NO: 1 is deficient. Such a polynucleotide may be used for recombinant production of ALS2 modified protein or for diagnosis of ALS2.

A primer set for a PCR amplification of ALS2, including various regions having base deficient sites in mutated ALS2 is (for example) a pair of synthetic oligonucleotides comprising base sequences of SEQ ID NO: 6 and NO: 7. This primer set is capable of a PCR amplification of the region (339 bp) including exon 3 and introns before and after that in the ALS2 gene. Another primer set may be composed of synthetic oligonucleotides comprising base sequences of SEQ ID NO: 8 and NO: 9 and is capable of PCR amplification of exons 2-4 (302 bp) of the ALS2 gene using RNA as a template. Any PCR product not cleaved by the restriction enzyme NarI is derived from the normal ALS2 gene but PCR products derived from a mutated ALS2 gene may be cleaved by NarI to give two fragments (FIG. 2c).

A recombinant vector of this invention may be a cloning vector or an expression vector. Vectors will be constructed depending upon the type of the polynucleotide as an insert or upon the object for use. For example, when an ALS2 protein or a modified protein thereof is produced using cDNA or its ORF region as an insert, there may be used an expression vector for an in vitro transcription or an expression suitable for each of prokaryotic cells such as Escherichia coli and Bacillus subtilis and eukaryotic cells such as yeast, insect cells and mammalian cells. When a genomic DNA of the ALS2 gene or a mutated gene thereof is used as an insert, it is also possible to use a BAC (bacterial artificial chromosome) vector or a cosmid vector. Such recombinant vectors are also useful, for example, as probes for diagnosis of chromosome abnormality by hybridization including fluorescent in situ hybridization (FISH). Further, a nucleic acid derived from a normal ALS2 gene may be recombined in a virus vector such as adenovirus or the like and the product may be used for genetic therapy.

In the manufacture of ALS2 peptide (including protein), a transformed cell of this invention may be a prokaryotic cell such as Escherichia coli and Bacillus subtilis or an eukaryotic cell such as from yeast, insects, and mammals. In addition, cells (such as blood stem cells) derived from a patient suffering from ALS2 which are transformed by a virus vector of this invention in which a nucleic acid derived from a normal ALS2 gene is recombined, may be used for a genetic therapy of ALS2. Such transformed cells may be prepared by introducing a recombinant vector into cells by means of known methods such as electroporation, calcium phosphate method, liposome method and DEAE dextran method.

A peptide of this invention may be an expression product of a normal ALS2 gene or an expression product of a mutated ALS2 gene. The normal gene product is a GTPase transcription factor or GEF having an amino acid sequence of SEQ ID NO: 2. Peptides of this invention are useful as immunogens for the preparation of an antibody, as target molecules for the development of therapeutic agents for ALS2, etc. These peptides may be prepared by methods involving isolating peptides from the cells of healthy persons or patients suffering from ALS2. Methods of chemical synthesis on the basis of a desired amino acid sequence from SEQ ID NO:2 or SEQ ID NO:3, etc. and (preferably) by production and isolation or purification from the above-mentioned transformed cells. Such transformed cells are incubated and isolation and purification are carried out for the culture by, for example, means of treatment with a modifier such as urea or with a surface-active agent, ultrasonic wave treatment, enzymatic digestion, precipitation by salting out or by solvent, dialysis, centrifugal separation, ultrafiltration, gel filtration, SDS-PAGE, isoelectric electrophoresis, ion exchange chromatography, hydrophobic chromatography, affinity chromatography and reversed phase chromatography. Such proteins may include fused proteins with any other protein. For example, fused proteins with glutathione-S-transferase (GST) or green fluorescent protein (GFP) may be exemplified. In addition, the protein expressed in cells may be subjected to various kinds of modifications in the cells after being translated. Accordingly, modified proteins are also included in the coverage of the protein of this invention. Examples of the modification after translation as such are elimination of N-terminal methionine, N-terminal acetylation, addition of sugar chain, limited decomposition by intracellular protease, myristoylation, isoprenylation and phosphorylation.

An antibody of this invention is a polyclonal antibody or monoclonal antibody which recognizes a peptide of this invention. Examples include an antibody prepared using a peptide comprising an amino acid sequence of continuous 5 amino acid residues or more of the first to the 46th amino acid sequence in SEQ ID NO: 2 as an antigen and an antibody prepared using a peptide comprising an amino acid sequence of continuous 5 amino acid residues or more of the 47th to the 1657th amino acid sequence in SEQ ID NO: 2 as an antigen. When those two kinds of antibodies are used, it is possible to detect and differentiate normal and A261del mutant proteins. The antibody of this invention includes all molecules which are able to bind to an epitope of an ALS2 protein or other peptide of this invention, and all of Fab, F(ab′)₂, Fv fragments, etc. thereof. Such an antibody can be obtained from serum after an animal is immunized using ALS2 derived protein or peptide as an antigen. Alternatively, the above expression vectors for eukaryotic cells may be introduced into muscle or skin of animals by injection or particle gun and then serum is collected therefrom. Examples of animals that may be used are mouse, rat, rabbit, goat, chicken, etc. When B cells collected from the spleen of an immunized animal are fused with myeloma cells to produce a hybridoma, it is possible to produce monoclonal antibodies.

The diagnostic method of this invention is one in which an ALS2 mutated gene or a transcription product of an ALS2 mutated gene is detected whereby the risk of onset of ALS2 may be estimated. Particularly amenable are persons of known ALS2 families although diagnosis is not limited thereto.

Genomic DNA of a person to be diagnosed may be subjected to a PCR amplification using any of the above-mentioned primer sets or other oligonucleotides of this invention. The resulting DNA fragment may be treated with one or more restriction enzymes such as NarI, and the person to be diagnosed where the DNA fragment is cleaved into fragments different from cleaving product produced from a person not suffering from ALS2 is indicative of a patient suffering from ALS2 or a person with some risk of ALS2 in view of the presence of a mutation in the ALS2 gene.

It is also possible to detect the ALS2 mutated genes by (for example) an allele-specific oligonucleotide probe method, an oligonucleotide ligation assay method, a PCR-SSCP method, a PCR-CFLP method, a PCR-PHFA method, an invader method, an RCA (rolling circle amplification) method and a primer oligo base extension method.

In detecting transcription products of ALS2 mutated genes, diagnosis may be carried out by determining the sequence of mRNA of the person to be diagnosed or cDNA thereof. It is also possible to carry out the diagnosis in such a manner that an ALS2 gene of a person to be diagnosed or cDNA thereof is recombined with an expression vector, transfected to cells and the expression product thereof measured.

Expression products of normal and mutant ALS2 genes may be assessed by measurement of molecular weight. For example, the frame shift caused by deletion of one base in normal ALS2 gene whereupon the modified protein is changed to a low-molecular protein (SEQ ID NO: 3) comprising the first to the 46th amino acid residues of SEQ ID NO: 2 and three amino acid residues (Pro-Ser-Glu) newly coded by the frame shift results in a product having a molecular weight easily comparable to naturally occurring gene products of the normal ALS2 gene. Further, diagnosis may be also carried out by the above antibody provided by this invention in which the ALS2 modified protein reacts with an antibody recognizing (for example) the first to the 46th amino acid sequence in SEQ ID NO: 2 or a region comprising amino acids 43-49 of SEQ ID NO:3, but does not react with an antibody recognizing the 47th to the 1657th amino acid sequence region in SEQ ID NO: 2. Antibodies specific for amino acids 476 to 545 of SEQ ID NO:84 as compared to any of the amino acids of SEQ ID NO:2 could be similarly used for diagnosis of the AG1548del. Diagnosis using antibodies may, for example, be carried out with an ELIZA method.

The mouse ALS2 gene of this invention is a mouse genomic gene isolated as a homolog of the human ALS2 gene and which codes for a mouse ALS2 protein comprising an amino acid sequence of SEQ ID NO: 5. Its CDNA has a base sequence of SEQ ID NO: 4. This gene may be used for the preparation of a “knock-out” mouse.

Such a “knock-out” mouse can be prepared by known gene targeting methods (Science, 244: 1288-1292, 1989) or generally according to the following example.

First, a DNA fragment of the mouse ALS2 gene including the initiation codon of the gene is modified whereupon a defective DNA fragment which deletes expression of the ALS2 gene is obtained. This defective DNA fragment is used for the preparation of a targeting vector for introduction of the modification into a mouse totipotent cell (ES cell) according to known methods (such as the method described in Science, 244: 1288-1292, 1989). For example, genomic DNA comprising the ALS2 gene is substituted or inserted with a resistant gene to a cytotoxin to prepare a recombinant plasmid DNA possessing the defective gene having a sequence homologous to the genomic DNA of the ALS2 gene at both terminals (the targeting vector). It is also possible for the resistant gene to be connected to a sequence such as PGK1 promoter and PGK1 polyadenylation signal for controlling the expression. It is preferred that the genomic DNA site of the ALS2CR6 gene which is substituted with or inserted by resistant gene be a genomic DNA region containing an exon region containing an initiation codon.

There are no particular limitations on such target vectors except that it will have a sequence which is homologous to genomic DNA of the ALS2 gene and a resistance sequence or other sequence useful for cell sorting (such as diphtheria toxin A gene and thymidine kinase gene of herpes virus). A promoter and enhancer may be appropriately combined and used. The targeting vector is then introduced into an ES (embryonic stem) cell according to known methods (e.g. Nature, 292: 154-156, 1981). Such methods include electric pulse, a liposome and calcium phosphate. When recombination efficiency of the gene to be introduced is of concern, the electric pulse methods is preferred. DNA in each of the ES cells into which gene is introduced is extracted and, by means of a southern blot analysis or a PCR assay, cells are selected in which a homologous gene recombination has taken place between the wild type ALS2 gene existing on the chromosome and the introduced defective ALS2 gene fragment resulting in placement of the defective gene fragment in the chromosome.

An ES cell having a defective gene prepared above may be injected into a blastocyst of a wild type animal and chimera-embryos obtained which are transplanted to the uterus of a preliminary parent. Resulting progeny are selected for the ALS2 defective gene and bred. Selection may be carried out by checking the difference in the color of hair or by extraction of DNA from a part of the body (such as the tail end) followed by conducting a southern blot analysis, a PCR assay after extraction of DNA, etc. As to the offspring obtained by a crossbreeding of animal of a wild type with a chimera animal where the ALS2 defective gene is in the generative cells, a southern blot analysis, a PCR assay or the like may be carried out using the DNA extracted from a part of the body (such as the tail end) as a material to identify a heterozygote into which the ALS2 defective gene is introduced. A heterozygote possessing the ALS2 defective gene which is stable in all generative cells and somatic cells may be bred to produce progeny in which the ALS2 gene is completely “knocked-out”.

An animal prepared as such may be used for analysis of function of ALS2 gene in onset of ALS2 and for screening of therapeutic drugs or development of therapeutic methods as an ALS2 model animal.

Methods and results of procedures carried out for cloning of the ALS2 gene and for functional analysis thereof are shown.

1. Methods

1-1. ALS2 Family

Sixteen cases including 8 individuals suffering from the disease obtained from a Tunisian consanginous ALS2 family (literature 2) were analyzed. The characteristic of ALS2 is a progressive convulsion of muscles of the limbs and the face accompanied by distal myoatrophy of the hand and the foot. Age of onset is between 3 and 10 years age (literature 2). According to biopsy of nerves and muscles and also to electromyography test, there was confirmed deletion of distal motor neuron (literature 2). When a gene type of the polymorphic DNA markers was analyzed together with clinical test data, ALS2 was clearly an autosomal recessive inheritance.

1-2. Transcription Map

Genome Data Base (GDB) (http://www.gdbwww.gdb.org) and UniGene (http://www.ncbi.nlm.nih.gov) of the Biotechnology Information Center (NCBI) which were open to the public for discriminating the sequence of transcribed DNA mapped within an objective region were retrieved. Sequence of genomic DNA overlapped with the objective region of ALS2 was retrieved from the “nr” or “htgs” data base of GenBank and utilized as the object for the test when a BLAST retrieval to the dbEST data base is conducted. In order to isolate the transcript of a full length, there were carried out RT-PCR, 5′-RACE and cDNA library screening. In addition, EST clone was purchased from Research Genetics and sequencing for DNA was carried out for measuring the insertion of the whole clone. Sequence of double stranded DNA was determined by conducting a dideoxy sequencing using a BigDye Terminator Cycle Sequencing Kit (ABI) and an AB1377DNA sequencer. All sequences of EST data, PCR products and DNA obtained from cDNA clone were determined and an estimated independent transcription unit was established. Then each unit was mapped on a physical map by a PCR method.

1-3. Identification of Exon

In order to determine the constitution of intron and exon of the transcription DNA, genomic DNA sequence data open to the public in GenBank data base was compared with the sequence of cDNA using a Sequencer Version 3.0 (Gene Codes Corporation) program according to the descriptions of BLAST (literature 28) and literatures (5 and 6).

1-4. PCR

Exon and intron/exon boundaries were subjected to a PCR amplification. ExTaq polymerase (Takara) was used and a cycle of 95° C. for 15 seconds, 60° C. for 30 seconds and 72° C. for 30 seconds was repeated for 35 times whereby about 50 mg of genomic DNA were amplified by a PCR. In order to detect the deficient form of the transcription DNA, an RT-PCR was carried out. Total RNA from lymphocytes of four patients of a family of ALS2 and two carriers was isolated. Total RNA extracted from a healthy human brain was purchased from Clontech. An RT-PCR was carried out using a SuperScript pre-amplification system (Gibco-BRL) according to the protocol of the manufacturer. The oligonucleotide primer for such a PCR was designed using Primer 3.0 (http://www-genome.wi.mit.edu). Table 1 lists the primers used for amplification of ALS2 (ALS2CR6).

TABLE 1

| | | |
|---|---|---|
| Pro- | SEQ | SEQ |

| | | duct | | ID | | ID | Re- |
|---|---|---|---|---|---|---|---|
| Exon | I.D. | (bp) | Forward Primer | NO | Reverse Primer | NO | marks |

| | | | | | | | |
|---|---|---|---|---|---|---|---|
| 1 | CALS370ex01 | 319 | 5′-GGAGAGACTGTGCTCCCAAG-3′ | 10 | 5′-AGCCCTCCTAGCCAATAGC-3′ | 11 | |
| 2 | CALS370ex02 | 381 | 5′-TAAGCTTAGTGGGCAGGCTC-3′ | 12 | 5′-TTCCCACTTAACAACCATCAAC-3′ | 13 | |
| 3 | CALS370ex03 | 339 | 5′-CCTAGTCATCCATGTGCTGG-3′ | 6 | 5′-TCCCATACCTGACCTTCCAC-3′ | 7 | |
| 4 | CALS370ex04-1 | 424 | 5′-CCAATTTGGTTAAATCTATAGGGG-3′ | 14 | 5′-GACAATGCCAGAGTGTGCTC-3′ | 15 | part |
| | | | | | | | of |
| | | | | | | | exon |
| | CALS370ex04-2 | 435 | 5′-CCAGCCCTTTGTTAGCAGTC-3′ | 16 | 5′-CTTCTTCCTGCCTGTCAAGG-3′ | 17 | part |
| | | | | | | | of |
| | | | | | | | exon |
| | CALS121 | 698 | 5′-TTGTACAATGCCTCCCTTCC-3′ | 18 | 5′-AGCCCAACATGACACCTTC-3′ | 19 | part |
| | | | | | | | of |
| | | | | | | | exon |
| 5 | CALS111ex002 | 490 | 5′-GATTGCTTGTTGCATAAGGG-3′ | 20 | 5′-ATACAGCATGCGATGTCAGG-3′ | 21 | |
| 6 | CALS120 | 322 | 5′-CTGGACTCCCACTCCTTCAC-3′ | 22 | 5′-GCTAGAAGAGCCCAGATTTCC-3′ | 23 | |
| 7 | CALS111ex004 | 443 | 5′-TGACTTTGTGTGCCTGTGTG-3′ | 24 | 5′-ATACCCTGGAAAATCTGGGG-3′ | 25 | |
| 8 | CALS111ex005 | 373 | 5′-TTTGCGCATTATCTCTGGTC-3′ | 26 | 5′-GTACGTATGAAATTCCCCCG-3′ | 27 | |
| 9 | CALS111ex006 | 389 | 5′-TTCCGTCTTACTCCTGCACC-3′ | 28 | 5′-GCCTTAGGATCCAATTCCTG-3′ | 29 | |
| 10 | CALS111ex007 | 467 | 5′-CAATGATGTACTGATGAACCAGC-3′ | 30 | 5′-CCTGATGGTTTAATGGTGGG-3′ | 31 | |
| 11 | CALS111ex008 | 348 | 5′-GCACATGGCAACAGGTTAAG-3′ | 32 | 5′-TCCTTGGCAGAATAACCGTG-3′ | 33 | |
| 12 | CALS111ex01 | 426 | 5′-CCCCTACCACTCCCTTTACC-3′ | 34 | 5′-CCAGTGGCTAATAGTACCTGTCC-3′ | 35 | |
| 13 | CALS111ex02 | 480 | 5′-TGGATGCATGATTCATTTCC-3 | 36 | 5′-TCCTTGGCTTTCCAAATGTC-3′ | 37 | |
| 14 | CALS111ex03 | 462 | 5′-CTATCCTGGGGTCTCTGCTG-3′ | 38 | 5′-TGCTATCGAAATGGTTGCTG-3′ | 39 | |
| 15 | CALS111ex04 | 290 | 5′-AGCTACGACCAGCAAATTCC-3′ | 40 | 5′-ATAGGGGTCCACCTTTCAGG-3′ | 41 | |
| 16 | CALS111ex05 | 454 | 5′-AAGGGGATATGGGCAGAGTC-3′ | 42 | 5′-AAATGCTTGCTTGGTTTTGG-3′ | 43 | |
| 17 | CALS111ex06 | 340 | 5′-AAAGGGCATCTTCATTGCAC-3′ | 44 | 5′-CACAAGAGGCAGAAAGAGCC-3′ | 45 | |
| 18 | CALS111ex07 | 298 | 5′-AATGCTTGATGAATTGTTGCC-3′ | 46 | 5′-ATGATCATCCTCACCCCAGG-3′ | 47 | |
| 19 | CALS111ex08 | 388 | 5′-TTGAAGATTTATGCCTGGGG-3′ | 48 | 5′-TGAGGTCACACGGCTATCAG-3′ | 49 | |
| 20 | CALS111ex09 | 379 | 5′-GTGTAGTGGGGCTGATGTCC-3 | 50 | 5′-TGGCTATGCAAACATTCAGG-3′ | 51 | |
| 21 | CALS111ex10 | 414 | 5′-AATGCAAAATACCACACATGG-3 | 52 | 5′-TCATTGGCTTAAACTGTGGG-3′ | 53 | |
| 22 | CALS111ex11 | 450 | 5′-CAACCTAGGGTTGATGCCTG-3′ | 54 | 5′-CATCTTCGGAAAGCAAAACC-3′ | 55 | |
| 23 | CALS111ex12 | 205 | 5′-CTTTGGGGATATGACTGCGT-3′ | 56 | 5′-GTAAAAGAATTTAGGGAGAAAAA-3′ | 57 | |
| 24 | CALS111ex13 | 260 | 5′-TTCCTCTAACCCCACATTTTATTC-3′ | 58 | 5′-TGCTTTTAAAATATTAACCAGCTTTG-3′ | 59 | |
| 25 | CALS122exe01 | 320 | 5′-TCAGTCTTGGCAGTTTTGGTC-3′ | 60 | 5′-CTGCTGTATGTTGAGCAGGTG-3′ | 61 | |
| 26 | CALS122exe02 | 455 | 5′-TGGATGCTCCACTTTGACTG-3′ | 62 | 5′-TTAAGAACCCCCTTGAGTGC-3′ | 63 | |
| 27 | CALS122exe03 | 362 | 5′-TTCCTGGTCCCAAAATTGAC-3′ | 64 | 5′-CAGGGTGAAACTACCCAAGC-3′ | 65 | |
| 28 | CALS122exe04 | 270 | 5′-TTTTATGCTTTTCAACCCCC-3′ | 66 | 5′-ACACACTTTCTCGCTGGGAC-3′ | 67 | |
| 29 | CALS122exe05 | 394 | 5′-TGATCTGAGCACAAAGGCTG-3′ | 68 | 5′-TAAACAGCGGTGGGTAGAGC-3′ | 69 | |
| | CALS122ex05 | 285 | 5′-AATGCTCCTTTTCTCCCACTC-3′ | 70 | 5′-TGCCAAATTTCCAATAATGC-3′ | 71 | |
| 30 | CALS122ex06 | 400 | 5′-TAATGGGGACAAGGAAGCC-3′ | 72 | 5′-GCTGAGGCAAAACAAGCATC-3′ | 73 | |
| 31 | CALS122ex07 | 375 | 5′-CCAAAGACCTGCACTCTGAC-3′ | 74 | 5′-CTGGCTTGGCTCTCTCCTAC-3′ | 75 | |
| 32 | CALS122ex08 | 312 | 5′-AAAAAGCACGATCAAATGGC-3′ | 76 | 5′-GGAAGAGCGTACTCCTGCTG-3′ | 77 | |
| 33 | CALS122ex09 | 393 | 5′-GCAGGAGTACGCTCTTCCAC-3′ | 78 | 5′-GAACAAAATGTGCTCTAAAGGC-3′ | 79 | |
| | CALS24exY | 486 | 5′-TCTTTTTCTCTCTGGGGCAG-3′ | 80 | 5′-TGCCTTCTGTGTTTTACCCTG-3′ | 81 | |
| 34 | CALS24exZ | 403 | 5′-GAAGGGAACAGGGAAAAGTG-3′ | 82 | 5′-TTACCTCCCTTTCAATCCTCC3′ | 83 | |

1-5. Analysis of Mutation

In order to detect the mutation of the DNA sequence at the exon or the intron/exon boundary, a DNA sequence of a PCR product of exon was determined. The DNA sequence of a PCR product of exon was analyzed using the same oligonucleotide as a primer. The sequence in the data base open to the public was compared with the DNA sequence obtained from patients, carriers and healthy persons and changes in the nucleotide were discriminated.

It was also confirmed that a new NarI site was formed (A261del) after the treatment with NarI by means of an RT-PCR amplification of exons 2-4 or a PCR amplification of exon 3. As to the primers for exon 3-PCR, there were used 5′-CCTAGTCATCCATGTGCTGG-3′ (SEQ ID NO: 6) and 5′-TCCCATACCTGACCTTCCAC-3′ (SEQ ID NO: 7). As to the primers for the RT-PCR of exons 2-4, there were used 5′-CTTGATAGACTTTCTGTAAAGAAG-3′ (SEQ ID NO: 8) and 5′-GGCTACTTGGACAAATCTCCACTG-3′ (SEQ ID NO: 9). Decomposed product with NarI was separated by 1.5% agarose gel.

1-6. Northern Blot Analysis

Northern (MTN) blot (Clontech) of many human adult tissues was hybridized with exon 4 labelled with ³²P-dCTP of ALS2CR6 or human β-actin cDNA in a Perfect Hyb hybridizing solution (Toyobo). The membrane was washed with 0.1×SSC containing 1% of SDS and subjected to an X-ray film (Bio-MAX, Kodak).

1-7. MRNA In Situ Hybridization

Antisense and sense cRNA probes were prepared from two mouse cDNA clones m2-as and m2-s. Those mouse cDNA clones covered a part of mouse mALS2CR6 cDNA (from the 1732nd to the 2685th bases of SEQ ID NO: 4; 954 bp) and inserted into pCR2.1 (Invitrogen) in an opposite direction. The probes were prepared according to the protocol of the manufacturer (Roche Molecular Biochemicals) by an in vitro transcription reaction where digoxigenin-labelled UTP and T7 polymerase were mixed. Preparation of the sample and method for the in situ hybridization were in accordance with the literature (29).

1-8. Retrieval of the Data Base

Each of DNA and amino acid sequences was compared with the data base of sequences of nucleotide and protein which were not overlapped each other using BLASTN and BLASTP. Domain and motive of protein were identified by MOTIF servers of Genome Net Japan (http://www.genome.ad.jp), search launcher of BCM (http://www.hgse.hem.tmc.edu/Search.launcher) and CD search of NCBI (http://www.hcbi.nlm.nih.gov).

2. Results

The inventors have prepared a physical map on the basis of YAC/BAC/PAC of genomic region of 3 Mb covering a complete candidate region to ALS2 (literatures 5 and 6). Sequences of EST and CDNA clone were analyzed within a broad area and, at the same time, this physical map was used for the mapping of 43 independent transcription units including previously analyzed 18 genes (KIAA0005, CLK1, PP1L3, ORC2L, NDUFB3, CFLAR, CASP10, CASP8, FZD7, NOP5, UBL1, BMPR2, FLJ10881, LOC57404, AIP-1, CD28, CTLA4 and AILIM) and new 10 full-length transcription products (ALS2CR1, ALS2CR2, ALS2CR3, ALS2CR4, ALS2CR5/MPP4, ALS2CR6, ALS2CR7, ALS2CR8, ALS2CR9 and ALS2CR12). Those genetic sequences were present in the locus of ALS2 (FIG. 1).

Juvenile ALS2 is rare and has a sign that, in teens and twenties, muscular convulsion of limbs, face and throat gradually expresses. Since ALS2 is recessively hereditary, it is predicted that this ALS2 disease may take place by a loss of a functional mutation. Big deletion or translocation in the ALS2 locus was investigated by a mapping of STS/EST content on the basis of a PCR and a southern blot analysis but that was not detected. After that, small deletion or base substitution in exon or intron-exon boundary was investigated. In order to detect those mutations, each gene was analyzed and an intron/exon boundary thereof was determined. Until now, 395 exons have been identified from 42 genes. In order to amplify exon and flanking sequence thereof including consensus sequence to splicing donor and acceptor, 411 primers in total were designed and those primers were used to amplify the genomic DNA of 10 normal control persons who were not related to 14 persons of the ALS2 family (FIG. 2a) by PCR. Sequence of each of those PCR products was determined whereby 77 sequence polymorphs in total of intron or exon were identified. Among those 77 polymorphs, 8 mutations contained in 4 different genes were related to ALS2 (Table 2).

TABLE 2

| Gene | Region | Normal | ALS2 |
|---|---|---|---|
| NOP5 | intron 2 | tatctc(T)9aattct | → (T)6 |
| NOP5 | intron 6 | gttttg(TTG)2ttttta | → (TTG)3 |
| ALS2CR6 | intron 2 | ggtaaAtcattt | → G |
| ALS2CR6 | exon 3 | gcaggcAgccctc | → A261 deletion* |
| ALS2CR8 | intron 6 | gtcagtAttataa | → G |
| ALS2CR9 | exon 4 | ctccagCatggac | → T (3rd codon) |
| ALS2CR9 | intron 7 | ttgggaTtttttt | → A |
| ALS2CR9 | intron 8 | aaaataCggatat | → T |

Among those sequence mutations, one nucleotide deletion (A261del) noted in exon 3 of ALS2CR6 broke the reading frame and it is suggested that such a mutation mutates the protein. All of the suspicious hetero-conjugative carriers show a duplicated sequence pattern starting from the first nucleotide after the deficient part (FIG. 2b). This deletion clearly moves together with an ALS2 expression type (FIG. 2c) and is not noted in 533 normal control individuals of various races (data not shown). In other mutations, one base substitution from C to T in exon 4 of ALS2CR9 gene is included (C873T). However, this mutation corresponds to the third codon and, therefore, it does not change the amino acid residue. In order to detect a splicing error which is made latent or manifest by other sequence mutation, an RT-PCR was carried out using total RNA extracted from lymphocytes of patients and healthy control persons but no sequence mutation of mRNA was detected (data not shown). Accordingly, the mutation related to ALS2 of intron or exon region does not cause a splicing error. From those results, it has been confirmed that deletion of one base in exon 3 of ALS2CR6 (A261del; Table 1) is mutation concerning ALS2.

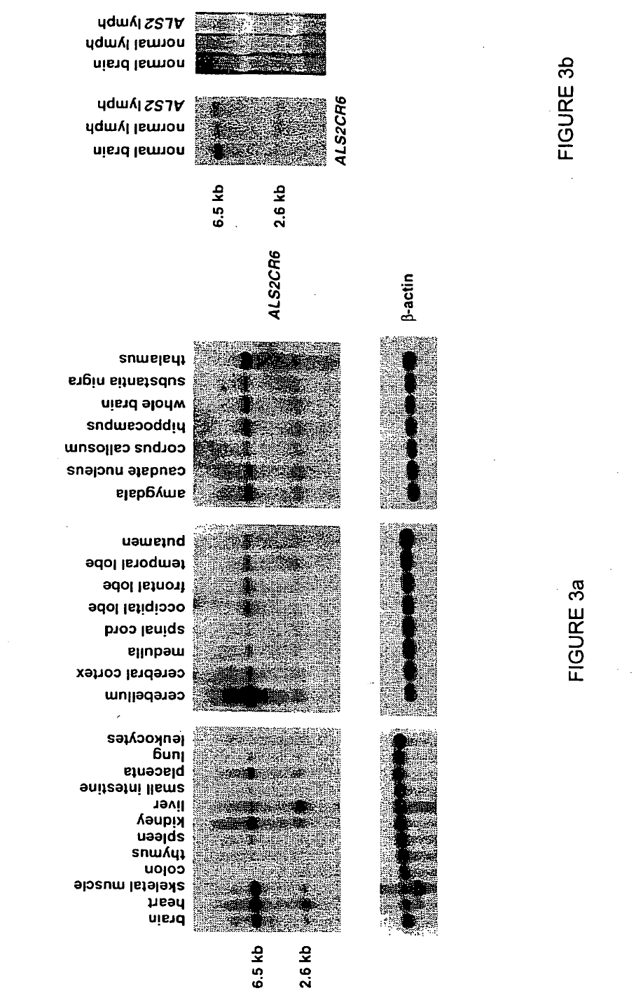

ALS2CR6 gene contains 33 introns and 34 exons and is present in a genomic DNA of 80.3 kb adjacent to a polymorphic DNA marker D2S2309 (FIG. 1). Transcription polarity of the ALS2CR6 gene is in the direction of central body from telomere. An ALS2CR6 transcription product (mRNA) has a full length of 6394 bp (SEQ ID NO: 1) having a single open reading frame (ORF) with a length of 4974 nucleotides (124-5,097 nt) and codes for a protein of 184 KDa comprising 1,657 amino acid residues. Polyadenylated estimated signal (AATAAA: 6,375-6,380 nt) and poly(A) region are clear. A short ALS2CR6 transcription product in a full length of 2,651 bp having 1,191 bp ORF coding for a 396 amino acid sequence was identified as well. This short variant splices a 5′-donor site after exon 4 and, as a result, a stop codon is formed after 25 amino acid residues of intron 4. Being correspondent to those results, 2 transcription products of about 6.5 kb and 2.6 kb were identified in many adult human tissues by a northern blot analysis (FIG. 3a). Except the liver where short transcription products are mostly expressed, both transcription products showed the similar expression pattern. It has been confirmed that a big transcription product of 6.5 kb is expressed in a slightly higher level than a transcription product of 2.6 kb and is most abundantly expressed in the cerebellum. This gene has been also confirmed to be expressed in cells of ALS2 patients (FIG. 3b).

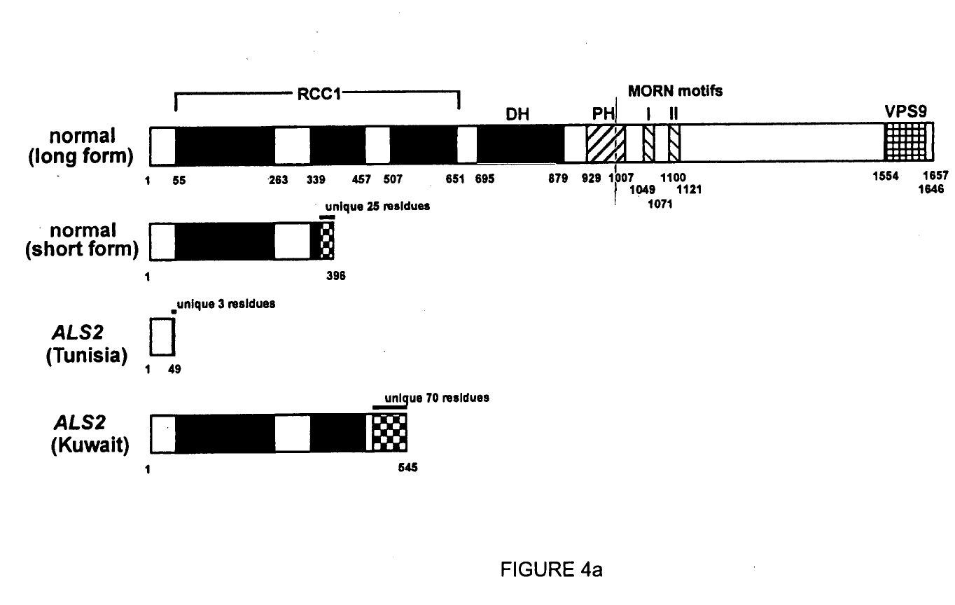

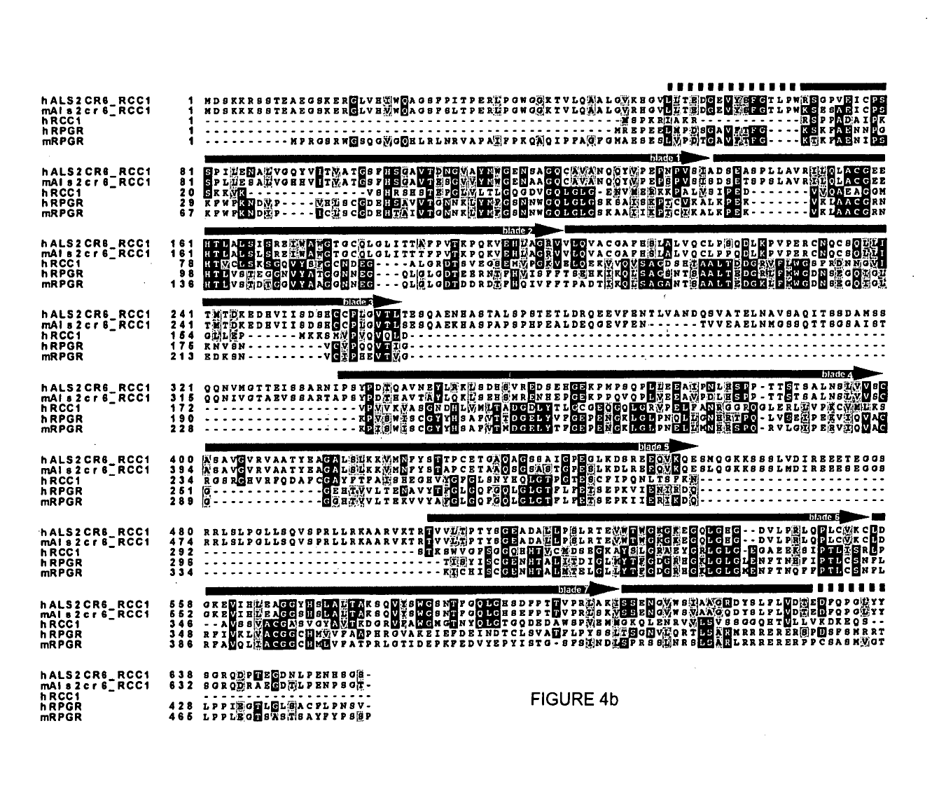

Further, a mouse homolog of ALS2CR6 was isolated and named mALS2CR6. A transcription product of mALS2CR6 is in a full length of 6,349 bp (SEQ ID NO: 4) having one ORF of 4,956 bp (124-5,076 nt) and codes for a protein of 183 kDa comprising 1651 amino acids (SEQ ID NO: 5). The ORF as a whole is well reserved in a DNA level (87% same) and a protein level (91% same; 94% similar; FIG. 4) between human being and mouse and it is suggested that ALS2CR6 gene is a gene which is well reserved in mammals.

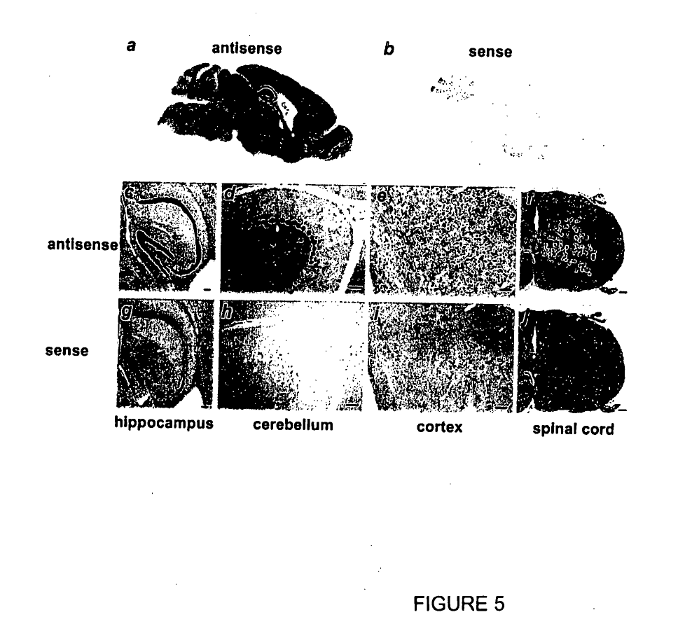

In order to check the localization property of expression of mALS2CR6 transcription product in the brain and the spinal cord of mouse, an in situ hybridization using riboprobe corresponding to a part of mALS2CR6 cDNA was carried out. The result was that, as shown in FIG. 5, the mALS2CR6 transcription products were expressed in various levels in nerve cells from the brain to the spinal cord, especially in neurons of hippocampal and dentate gyrus, cerebellar Purkinje cells, neurons of cerebral cortex and spinal cinerea including anterior horn cells. In addition, a significant expression was noted in neurons of olfactory bulb, basal nucleus and cranial nerve nucleus as well.

Human ALS2CR6 protein showed many interesting properties (FIG. 6a). The first property is present in a region of A-terminal side of ALS2CR6 and it showed a high homology to RCC1 (regulatory factor for concentrating the chromosome; literature 7) and RPGR (GTPase for pigmentary retinitis; literature 8)(FIG. 8). RCC1 and RPGR protein acts as a guanine nucleotide exchange factor (GEF) for GTPase like Ran. The second property is that ALS2CR6 has a Db1-homologous (DH) domain and a pleckstrin-homologous (PH) domain and both domains are typical domains noted in RhoGEF protein (literatures 9 and 10). In addition, VPS9 domain is noted in a C-terminal region as well. VPS9 domain is noted in many GEF including Vps9 (literature 11) and Rabex-5 (literature 12) and each is said to mediate the selection of vacuole protein and the phagocytic transportation. Two MORN motives comprising 14 amino acids (literature 13) were noted as well. According to the recent study for junctophilin containing an MORN motive, this motive is shown to contribute in bonding of plasma membrane (literature 13). It has been known that GEF is related to a GDP bonding form of GTPase and promotes the dissociation of GDP and bonding of GTP whereby GTPase is activated. Since it has been known (literatures 18 and 19) that GEF plays an important role in many signal transmission cascades (literature 14), neuron formation (literature 15), membrane transportation (literature 16) and formation of actin cell skeleton (literature 17), it is likely that ALS2CR6 acts as a regulatory factor/activator of Ran-related GTPase, regulates the formation of membrane and acts in a (membrane) transportation of cells including neurons.

According to an RT-PCR analysis, a transcription product of mutated ALS2CR6 gene is transcribed from chromosomes of the patient (FIG. 2c) and produces a modified protein comprising 49 amino acids having three new residues (Pro-Ser-Glu) at C-terminal (FIG. 6a). Since this modified protein has no functional domain corresponding to ALS2CR6 protein, it seems to make the inherent function lost. Accordingly, the A261del mutation noted in this ALS2CR6 is related to the fact that ALS2 is recessively hereditary.

A recent finding that ALS is related to defect for the transportation of axon and the formation of cell skeleton (literatures 20, 21 and 22) induces a hypothesis that ALS2CR6 gene corresponds to ALS2 and that ALS2 is generated by the defect of membrane structure due to lacking in a regulatory function of membrane structure Ran-related GTPase.

ALS2CR6 gene is the second ALS gene succeeding to the determination of role of copper-zinc superoxide desmutase (SDS-1) in ALS. Mutation of SOD-1 is related to the form of tardive autosomal dominance (literature 23).

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

### References

1. Siddique, T., Nijhawan, D.& Hentati, A. Molecular genetic basis of familiar ALS. Neurology 47, S27-S35 (1996).

2. Ben Hamida, M., Hentati, F. & Ben Hamida, C. Hereditary motor system diseases (chronic juvenile amyotrophic lateral sclerosis). Brain 113, 347-363 (1990).

3. Hentati, A. et al. Linkage of recessive familial amyotrophic lateral sclerosis to chromosome 2q33-q35. Nature Genet. 7, 425-428 (1994).

4. Hosler, B. A. et al. Refined mapping and characterization of the recessive familial amyotrophic lateral sclerosis locus (ALS2) on chromosome 2q33. Neuro genetics 2, 34-42 (1998).

5. Hadano, S. et al. A yeast artificial chromosome-based physical map of the juvenile amyotrophic lateral sclerosis (ALS2) critical region on human chromosome 2q33-q34. Genomics 55, 106-112 (1999).

6. Hadano, S. et al. Cloning and characterization of three novel genes, ALS2CR1, ALS2CR2, and ALS2CR3, in the juvenile amyotrophic lateral sclerosis (ALS2) critical region at chromosome 2q33-q34: candidate genes for ALS2. Genomics 71, 200-213 (2001).

7. Lerman-Sagie, T., Filiano, J., Smith, D. W. & Korson, M. Infantile onset of hereditary ascending spastic paralysis with bulbar involvement. J. Child Neurol. 11, 54-57 (1996).

8. Ohtsubo, M. et al. Isolation and characterization of the active cDNA of the human cell cycle gene (RCC1) involved in the regulation of onset of chromosome condensation. Genes Dev. 1,585-593 (1987).

9. Meindl, A. et al. A gene (RPGR) with homology to the RCC1 guanine nucleotide exchange factor is mutated in X-linked retinitis pigmentosa (RP3). Nature Genet. 13, 35-42 (1996).

10. Renault, L. et al. The 1.7 Å crystal structure of the regulator of chromosome condensation (RCC1) reveals a seven-bladed propeller. Nature 392, 97-101 (1998).

11. Carazo-Salas, R. E. et al. Generation of GTP-bound Ran by RCC1 is required for chromatin mitotic spindle formation. Nature 400, 178-181 (1999).

12. Soisson, S. M., Nimnual, A. S., Uy, M., Bar-Sagi, D. & Kuriyan, J. Crystal structure of the Db1 and pleckstrin homology domains from the human Son of sevenless protein. Cell 95, 259-268 (1998).

13. Hama, H., Tall, G. G. & Horazdovsky, B. F. Vps9p is a guanine nucleotide exchange factor involved in vesicle-mediated vacuolar protein transport. J. Biol. Chem. 274, 15284-15291 (1999).

14. Horiuchi, H. et al. A novel Rab5 GDP/GTP exchange factor complexed to Rabaptin-5 links nucleotide exchange to effector recruitment and function. Cell 90, 1149-1159 (1997).

15. Takeshima, H., Kamazaki, S., Nishi, M., Iino, M. & Kangawa, K. Junctophilins: a novel family of junctional membrane complex proteins. Mol. Cell 6, 11-22 (2000).

16. Barrett, K., Leptin, M. & Settleman, J. The Rho GTPase and a putative RhoGEF mediate a signaling pathway for the cell shape changes in Drosophila gastrulation. Cell 91, 905-915 (1997).

17. Martijn, F. B. G. et al. Identification of a novel, putative Rho-specific GDP/GTP exchange factor and a RhoA-binding protein: control of neuronal morphology. J. Cell. Biol. 137, 1603-1613 (1997).

18. Roepman, R. et al. The retinitis pigmentosa GTPase regulator (RPGR) interacts with novel transport-like proteins in the outer segments of rod photoreceptors. Hum. Mol. Genet. 9, 2095-2105 (2000).

19. Luo, L. Rho GTPases in neuronal morphogenesis. Nature Rev. Neurosci. 1, 173-180 (2000).

20. Hall, A. Rho GTPases and the actin cytoskeleton. Science 279, 509-514 (1998).

21. Culbertson, M. R. RNA surveillance. Unforeseen consequences for gene expression, inherited genetic disorders and cancer. Trends Genet. 15, 74-80 (1999).

22. Collard, J.-F., Côté, F. & Julien, J.-P. Defective axonal transport in a transgenic mouse model of amyotrophic lateral sclerosis. Nature 375, 61-64 (1995).

23. Williamson, T. L. & Cleveland, D. W. Slowing of axonal transport is a very early event in the toxicity of ALS-linked SOD1 mutants to motor neurons. Nature Neurosci. 2, 50-56 (1999).

24. Rosen, D. R. et al. Mutations in Cu/Zn superoxide dismutase gene are associated with familial amyotrophic lateral sclerosis. Nature 362, 59-62 (1993).

25. Chance, P. F. et al. Linkage of the gene for an autosomal dominant form of juvenile amyotrophic lateral sclerosis to chromosome 9q34. Am. J. Hum. Genet. 62, 633-640 (1998).

26. Blair, I. P. et al. A gene for autosomal dominant juvenile amyotrophic lateral sclerosis (ALS4) localizes to a 500-kb interval on chromosome 9q34. Neurogenetics 3, 1-6 (2000).

27. Hentati, A. et al. Linkage of a commoner form of recessive amyotrophic lateral sclerosis to chromosome 15q15-q22 markers. Neurogenetics 2, 55-60 (1998).

28. Hosler, B. A. et al. Linkage of familial amyotrophic lateral sclerosis with frontotemporal dementia to chromosome 9q21-q22. JAMA 284, 1664-1669 (2000).

29. Altschul, S. F. et al. Gapped BLAST and PSI-BLAST: a new generation of protein database search programs. Nucleic Acids Res. 25, 3389-3402 (1997).

30. Matsumoto, K. et al. Neuronal apoptosis inhibitory protein (NAIP) may enhance the survival of granulosa cells thus indirectly affecting oocyte survival. Mol. Repod. Dev. 54, 103-111 (1999).

What is claimed is:

1. An isolated nucleic acid that codes for a peptide having at least 75% identity to all of an amino acid sequence selected from the group consisting of: SEQ ID NO:2; SEQ ID NO:3; SEQ ID NO:5; SEQ ID NO:84; and, amino acids 372-1657 of SEQ ID NO:2.

2. The nucleic acid of claim 1 that codes for a peptide having about 80% or more sequence identity to the selected sequence.

3. The nucleic acid of claim 1 that codes for a peptide having about 85% or more sequence identity to the selected sequence.

4. The nucleic acid of claim 1 that codes for a peptide having about 90% or more sequence identity to the selected sequence.

5. The nucleic acid of claim 1 that codes a peptide having about 95% or more sequence identity to the selected sequence.

6. The nucleic acid of any one of claims 1-5, wherein the selected sequence is SEQ ID NO:2.

7. The nucleic acid of claim 1, wherein the selected sequence is SEQ ID NO:3.

8. The nucleic acid of claim 1, wherein the selected sequence is SEQ ID NO:5.

9. The nucleic acid of claim 1, wherein the selected sequence is SEQ ID NO:84.

10. An isolated nucleic acid consisting essentially of a nucleotide sequence having at least 75% identity to all of a nucleotide sequence or a complementary sequence thereof, selected from the group consisting of: SEQ ID NO:1; SEQ ID NO:4; nucleotides 124-5094 of SEQ ID NO:1; nucleotides 1225-5094 of SEQ ID NO:1; and, nucleotides 124-5076 of SEQ ID NO:4.

11. The nucleic acid of claim 10 having about 80% or more sequence identity to the selected sequence or complementary sequence thereof.

12. The nucleic acid of claim 10 having about 85% or more sequence identity to the selected sequence or complementary sequence thereof.

13. The nucleic acid of claim 10 having about 90% or more sequence identity to the selected sequence or complementary sequence thereof.

14. The nucleic acid of claim 10 having about 95% or more sequence identity to the selected sequence or complementary sequence thereof.

15. The nucleic acid of any one of claims 10-14, wherein the selected sequence is SEQ ID NO:1.

16. The nucleic acid of any one of claims 10-14, wherein the selected sequence is SEQ ID NO:4.

17. The nucleic acid of any one of claims 10-14, wherein the selected sequence is nucleotides 124-5094 of SEQ ID NO: 1.

18. The nucleic acid of any one of claims 10-14, wherein the selected sequence is amino acids 124-5076 of SEQ ID NO:4.

19. The isolated nucleic acid of any one of claims 1-18 joined to a second nucleic acid, wherein the second nucleic acid is not naturally associated with the isolated nucleic acid.

20. A recombinant vector comprising a nucleic acid according to any one of claims 1-19.

21. A cell comprising a nucleic acid of claim 19 or a vector of claim 20.

22. An oligonucleotide of 6 to 75 nucleotides, wherein the oligonucleotide hybridizes to a nucleic acid according to any one of claims 1-18 or a complementary sequence thereof, under stringent conditions.

23. The oligonucleotide of claim 22 of about 10 to about 40 nucleotides.

24. The oligonucleotide of claim 22 of about 15 to about 30 nucleotides.

25. The oligonucleotide of claim 22 of about 15 to about 25 nucleotides.

26. The oligonucleotide of any one of claims 22-25 capable of hybridizing under stringent conditions to a nucleic acid encoding a peptide consisting of the sequence of SEQ ID NO:3 or the complementary nucleic acid sequence thereof, but not to a nucleic acid encoding a peptide consisting of the sequence of SEQ ID NO:2 or the complementary nucleic acid sequence thereof.

27. The oligonucleotide of any one of claims 22-25 capable of hybridizing under stringent conditions to a nucleic acid encoding a peptide consisting of the sequence of SEQ ID NO:84 or the complementary nucleic acid sequence thereof, but not to a nucleic acid encoding a peptide consisting of the sequence of SEQ ID NO:2 or the complementary nucleic acid sequence thereof.

28. The oligonucleotide of any one of claims 22-27 joined to a label.

29. A kit comprising two or more different oligonucleotides according to any one of claims 22-27 for use in nucleic acid amplification.

30. An isolated peptide comprising a sequence of amino acids coded by a nucleic acid according to any one of claims 1-19 or a recombinant vector according to claim 20.

31. A peptide consisting essentially of a sequence of at least 5 contiguous amino acids from a sequence selected from the group consisting of: amino acids 146 of SEQ ID NO:2; amino acids 47-1657 of SEQ ID NO:2; SEQ ID NO:3; amino acids 43-49 of SEQ ID NO:3; SEQ ID NO:84; and amino acids 476 to 545 of SEQ ID NO:84.

32. A peptide comprising at least 5 contiguous amino acids from amino acids 4349 of SEQ ID NO:3 or amino acids 476 to 545 of SEQ ID NO:84.

33. An antibody which binds a peptide according to any one of claims 30-32.

34. The antibody of claim 33 prepared by using a peptide according to any one of claims 30-32 as an antigen.

35. A non-human mammal comprising a mutated gene, wherein the gene but for the mutation would encode a protein having at least 75% sequence identity to all of SEQ ID NO:2 or SEQ ID NO:5.

36. The mammal of claim 35, wherein the protein has at least 85% sequence identity to all of SEQ ID NO:1 or SEQ ID NO:2.

37. The mammal of claim 35 or 36, wherein the mutated gene does not express a protein having biological activity.

38. The mammal of claim 35, 36, or 37, wherein the mutated gene is incapable of expression of a protein.

39. The mammal of any one of claims 35-38, wherein the mammal is a rodent.

40. The mammal of claim 39, wherein the rodent is a mouse.

41. A method for the diagnosis of amyotrophic lateral sclerosis type 2 in a patient, comprising detecting the presence of a mutation in a gene that encodes a protein having at least 75% sequence identity to SEQ ID NO:2.

42. The method of claim 41, wherein the protein has at least about 90% sequence identity to SEQ ID NO:2.

43. The method of claim 41, wherein the protein has at least about 95% sequence identity to SEQ ID NO:2.

44. The method of claim 41, wherein the protein has at least about 97% sequence identity to SEQ ID NO:2.

45. The method of claim 41, wherein the protein has essentially the sequence of SEQ ID NO:2 but for the presence of the mutation.

46. The method of any one of claims 41-45, comprising detecting the presence of the mutation in a biological sample from the patient.

47. The method of any one of claims 41-46, wherein the detecting comprises comparing a sequence of the gene, a RNA transcript of the gene, and a cDNA made from the RNA transcript, or a protein expressed by the gene from a human patient, to SEQ ID NO:1, wherein a difference in sequence is indicative of mutation.

48. The method of claim 46, comprising contacting nucleic acids obtained from the biological sample or cDNA made from said nucleic acids, with one or more oligonucleotides according to any one of claims 22 to 28.

49. The method of claim 46, comprising detecting whether the one or more oligonucleotides hybridize to said nucleic acids or cDNA, under stringent conditions.

50. The method of claim 46, comprising amplification of nucleic acids or cDNA to which two or more of said oligonucleotides hybridize, and determining the presence of an amplified product.

51. A method for the diagnosis of amyotrophic lateral sclerosis type 2, comprising detecting the presence or absence of a protein having at least 85% sequence identity to all of SEQ ID NO:2 in a patient.

52. A method for the diagnosis of amyotrophic lateral sclerosis type 2, comprising detecting the presence or absence of a protein having at least 95% sequence identity to all of SEQ ID NO:2 in a patient.

53. The method of claim 51, wherein the detecting comprises determining whether a protein having at least 85% sequence identity to all of SEQ ID NO:2 is present in a biological sample from the patient.

54. The method of claim 52, wherein the detecting comprises determining whether a protein having at least 95% sequence identity to all of SEQ ID NO:2 is present in a biological sample from the patient.

55. A method for the diagnosis of amyotrophic lateral sclerosis type 2, comprising detecting the presence or absence of a protein having at least 85% sequence identity to all of SEQ ID NO:3 or SEQ ID NO:84 in a biological sample from the patient.

56. A method for the diagnosis of amyotrophic lateral sclerosis type 2, comprising detecting the presence or absence of a protein having at least 95% sequence identity to all of SEQ ED NO:3 or SEQ ID NO:84 in a biological sample from the patient.

57. The method of any one of claims 51-56, comprising contacting an antibody according to any one of claims 33 or 34 with a biological sample from the patient and determining whether the antibody binds to protein in the sample.

58. A method of treatment of amyotrophic lateral sclerosis type 2, comprising administering a peptide, a nucleic acid, or a pharmaceutical composition comprising the peptide or nucleic acid to a patient in need thereof, wherein the peptide comprises an amino acid sequence having at least 90% identity to SEQ ID NO:2 or a fragment thereof, and the nucleic acid codes for said peptide.

59. A method of treatment of amyotrophic lateral sclerosis type 2, comprising administering a peptide, a nucleic acid, or a pharmaceutical composition comprising the peptide or nucleic acid to a patient in need thereof, wherein the peptide comprises an amino acid sequence having at least 95% identity to SEQ ID NO:2 or a fragment thereof, and the nucleic acid codes for said peptide.

60. A method of treatment of amyotrophic lateral sclerosis type 2, comprising administering a composition to a patient in need thereof, wherein the composition mimics the biological activity of the peptide of SEQ ID NO. 2.

61. The use of a peptide or a nucleic acid for preparation of a medicament for treatment of amyotrophic lateral sclerosis type 2, wherein the peptide comprises an amino acid sequence having at least 90% identity to SEQ ID NO:2 or a fragment thereof, and the nucleic acid codes for said peptide.

62. The use of a peptide or a nucleic acid for preparation of a medicament for treatment of amyotrophic lateral sclerosis type 2, wherein the peptide comprises an amino acid sequence having at least 95% identity to SEQ ID. NO:2 or a fragment thereof, and the nucleic acid codes for said peptide.
